(19) United States
(12) Patent Application Publication
(10) Pub. No.: US 2026/0176268 A1
(43) Pub. Date: Jun. 25, 2026

(54) LIQUID CRYSTALLINE SAM VIA SPIN-COAT ASSEMBLY AND THEIR AREA SELECTIVE DEPOSITION PROPERTIES

(71) Applicant: Merck Patent GmbH, Darmstadt (DE)

(72) Inventors: Christoph Wetzel, Ehingen (Donau) (DE); Nicolas Blouin, Grez-Doiceau (BE); Rocco Fortte, Frankfurt Am Main (DE); Sachin Bobade, Branchburg, NJ (US); Durairaj Baskaran, Bridgewater, NJ (US)

(21) Appl. No.: 19/125,614

(22) PCT Filed: Nov. 9, 2023
(86) PCT No.: PCT/EP2023/081276

§ 371 Date: Apr. 29, 2025

Related U.S. Application Data
(60) Provisional application No. 63/383,374, filed on Nov. 11, 2022.

(51) Int. Cl. C07D 471/04; C07D 487/04; C09K 19/30

(52) U.S. Cl. CPC C07D 471/04; C07D 487/04; C09K 2019/3004

(57) ABSTRACT

Described is a patterned liquid crystal monolayer structure (100) on a patterned substrate (900), wherein said patterned substrate is comprising surface areas which grafted onto (500) by a liquid crystal compounds (300) as a self-assembled monolayer (SAM) (200) and surface areas which are not grafted onto (400) by this SAM on this patterned substrate. Also described, are compositions of select liquid crystal (200) with different types of polar anchor groups, in an organic spin casting solvent and the method of using this composition to selectively deposit a SAM on a substrate containing both metallic surface areas and non-metallic inorganic silicon compound surface areas on either one of these areas, depending on the nature of a polar anchor group on the liquid crystal compound, and using this selective SAM deposition as a barrier to selectively deposit a metal oxide dielectric by ALD on the area not grafted by a SAM.

### FIELD

The disclosed subject matter pertains to templates on IC substrates formed by a self-assembled monolayer (SAM) of liquid crystals with polar anchor groups which selectively form a SAM only on certain areas of the IC substrate, the compositions used to form these templates, and the method of forming these templates and using this selective SAM as a barrier to affect selective ALD deposition of dielectric metal oxides on areas of the IC substrate not protected by SAM.

### RELATED ART

In the last decades, many efforts have been made to further increase miniaturization, cost, speed, power consumption, and versatility concerning the silicon based integrated circuits industry (IC). Tremendous progress has been made to increase the numbers of transistors per CPU, and today microprocessors contain up to four billion transistors per small unit area. To build these kinds of processors, a series of operations is performed; among the process steps are photolithography, etching and deposition. Photolithography is a key step in the production of transistors and resistors. The main factor effecting resolution of the obtained structures is the illumination wavelength used during photolithography. The smaller the wavelength, the higher the possible resolution, and the smaller the pitch size. Currently, 193 nm is the smallest illumination wavelength that has been introduced in IC, and it can provide ˜80 nm pitch; however, the industry target today is to achieve pitch sizes of single nanometers for the same unit area.

To avoid the investment in new equipment and materials, multiple patterning photolithography techniques have been introduced, and have achieved pitches of ˜40 nm. But multiple patterning comes at the cost of the increased number of steps, defects, cost, process time, tools, fab space, consumable materials, and personnel.

In conventional lithography approaches, ultraviolet (UV) radiation may be used to expose through a mask onto a photoresist layer coated on a substrate or layered substrate. Positive or negative photoresists are useful, and these can also contain a refractory element such as silicon to enable dry development with conventional integrated circuit (IC) plasma processing. In a positive photoresist, UV radiation transmitted through a mask causes a photochemical reaction in the photoresist such that the exposed regions are removed with a developer solution or by conventional IC plasma processing. Conversely, in negative photoresists, UV radiation transmitted through a mask causes the regions exposed to radiation to become less removable with a developer solution or by conventional IC plasma processing. An integrated circuit feature, such as a gate, via or interconnect, is then etched into the substrate or layered substrate, and the remaining photoresist is removed. When using conventional lithographic exposure processes, the dimensions of features of the integrated circuit feature are limited. Further reduction in pattern dimensions is difficult to achieve with radiation exposure due to limitations related to aberrations, focus, proximity effects, minimum achievable exposure wavelengths, and maximum achievable numerical apertures. Directed self-assembly is a promising approach that has been of interest in overcoming some of the drawbacks of conventional lithography as outlined above. Directed self-assembly of block copolymers is a method useful for generating smaller and smaller patterned features for the manufacture of microelectronic devices in which the critical dimensions (CD) of features on the order of nanoscale can be achieved. Directed self-assembly methods are desirable for extending the resolution capabilities of microlithographic technology. The need for large-scale integration has led to a continued shrinking of the circuit dimensions and features in the devices. In the past, the final resolution of the features has been dependent upon the wavelength of light used to expose the photoresist, which has its own limitations. The most recent technique for achieving the targeted pitches using shorter wavelengths of light is Extreme Ultraviolet Lithography (EUV), which could theoretically achieve a maximum pitch resolution of ˜13.5 nm. However, this technique has high defectivity, which is not compatible with industry expectations. The defectivity specific to EUV is generalized as mask defectivity, and is a combination of substrate, multilayer blank, and absorber patterning defects. Additionally, this technique is especially high cost, with only 53 machines worldwide capable of production. Direct assembly techniques, such as graphoepitaxy and chemoepitaxy using block copolymer imaging, are highly desirable techniques used to enhance resolution while reducing CD variation. These techniques can be employed to either enhance conventional UV lithographic techniques or to enable even higher resolution and CD control in approaches employing EUV, e-beam, deep UV, or immersion lithography.

Directed self-assembly (DSA) of Block copolymer (BCP) lithography is an additional alternative or compliment to conventional lithography's, which differs from the methods mentioned above in that it involves a combination of bottom-up and top-down methods. Templates created with photolithography\EUV techniques (top-down methods) are spin-coated with the BCP, which is then made to phase separate under the influence of the guiding patterns (templates) at very high resolution of single nanometers (bottom-up). Afterwards, one block is selectively etched, and a desired pitch pattern is obtained in accordance with the size of the block.

In IC technology area selective deposition of organic or inorganic material is an important process in IC industry which requires selectivity exclusive to either metal or dielectric via direct and indirect assembly processes. One such application is passivation of dielectric or metal surfaces on given pattern substrates for area-selective deposition of metal oxide via atomic layer deposition (ALD). This application requires selective grafting of organic materials such as self-assembled monolayers (SAM) or chain end functional polymers (brush) for introducing a subsequent deposition or assembly process that enables passivation of selective area underneath. An easy method such as spin-coating organic materials that can exhibit selectivity to specific areas of a substrate in chip making lithographic processes is highly sought after in industry for ALD or DSA. Common methods of SAM deposition are solution or immersion method and vapor phase. Both these methods have its own merits and demerits. Chemistry and processing of SAM precursors limits its selective deposition via spin-coat method.

For liquid crystal display (LCD) technology orientation of liquid crystals (LCs) on surfaces of glass or indium tin oxide (ITO) is achieved by surface modification to enable orientation of LCs. This is achieved via surface modification with alkyl silanes with or without LC components and siloxy functional groups. In this approach Glass or ITO surfaces are modified with alkoxysilane SAM's using solution dip-coat method, and vapor phase delivery. Liquid crystal compounds (LCC) with functional groups like carboxylic acids, hydroxyl, amines, thiol are known and in liquid crystal display (LCD) technology are used as neat materials or as mixture with photo-curable polymer matrix.

Atomic layer deposition (ALD) is an important technique for depositing thin films for a variety of applications in the semiconductor field. It is advantageous because it offers precise thickness control at the Angstrom or monolayer level because of its self-limiting surface chemistry. Because of the increasing demands of the semiconductor roadmap which are testing the limits of the past electronic material ALD is increasingly important because of its ability to control deposition on the atomic scale and to deposit conformally on very high aspect ratio structures. For fabrication of 3-D devices, lateral patterning of ALD is used, for instance, by using conventional photoresist and photolithography like semiconductor processing, other masking polymer layers, direct-write electron beams to remove selectively a masking layer to allow ALD on that area or by using patterned octadecyl trichlorosilane monolayers.

There is a need for a simple spin coatable method compatible with standard IC track equipment which can easily form self-assembled monolayers (SAM) with a high density of moieties that can selectively protect metallic surface areas or non-metallic surface areas on an IC substrate to enhance atomic layer deposition of dielectric metal oxides in unprotected areas and is compatible with the nanometer-sized dimensions predicated by modern methods of IC patterning such as for example EUV, DSA and combinations of these techniques.

### DETAILED DESCRIPTION OF DRAWINGS

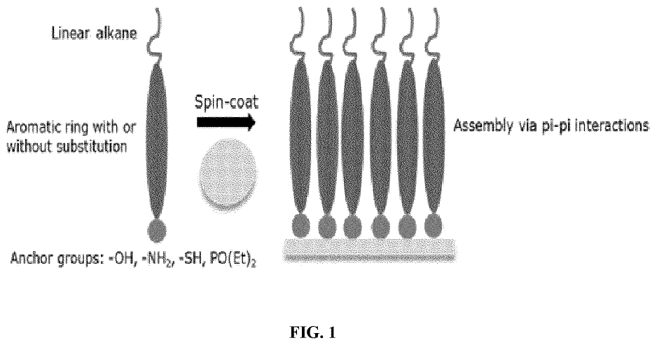

FIG. 1 Schematic of spin-on SAM assembly of liquid crystalline molecule

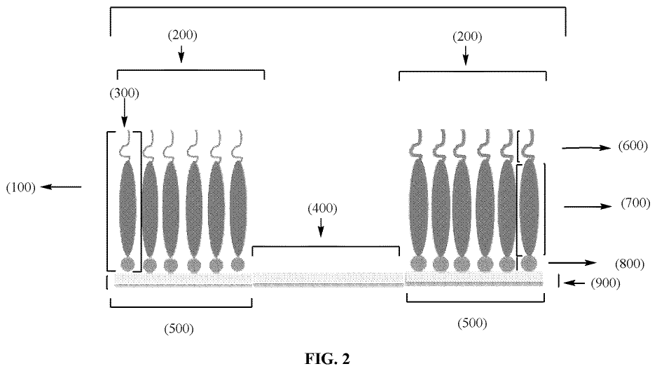

FIG. 2 Schematic depiction of a portion of a cross section taken perpendicularly to a patterned substrate in which only some portions of this substrate have an overlying SAM of a liquid crystal consisting of a linear alkyl group a central core with at least two phenyl moieties and a polar anchoring group.

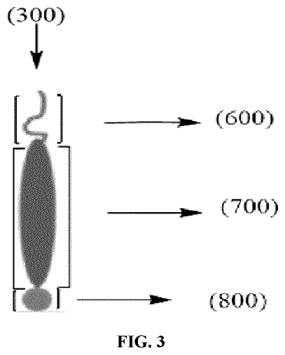

FIG. 3 Schematic depiction of liquid crystal compound (300).

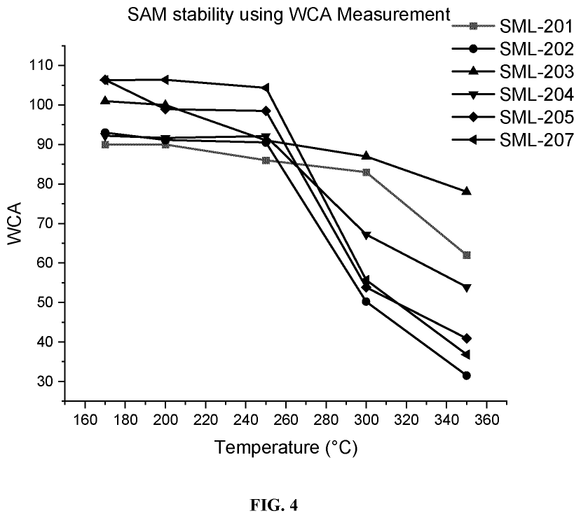

FIG. 4 Thermal stability of SAM via change in water contact angle (SAM's were heated for 5 min under nitrogen at different temperature).

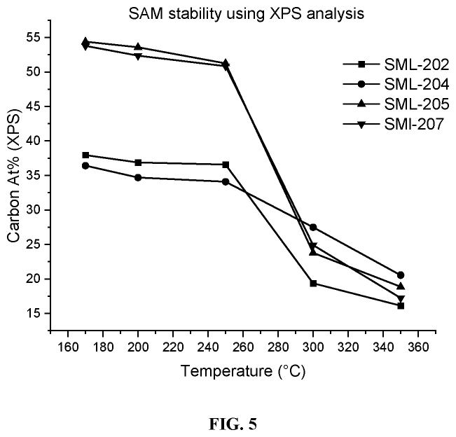

FIG. 5 SAM thermal via change in XPS carbon atomic percentage (SAM's were heated for 5 min under nitrogen at different temperature).

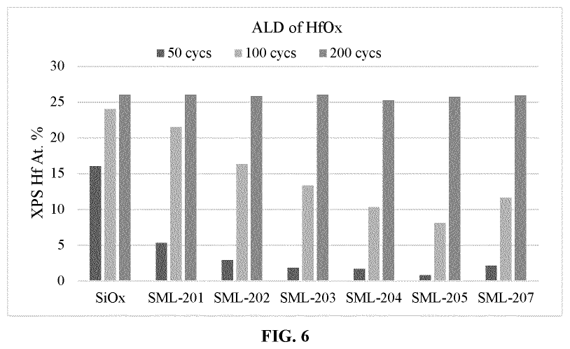

FIG. 6 Passivation properties of dielectric SAM's against atomic layer deposition of hafnium oxide at 300° C.

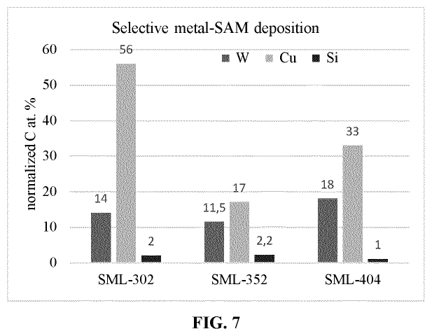

FIG. 7 SAM selectivity characterization using XPS.

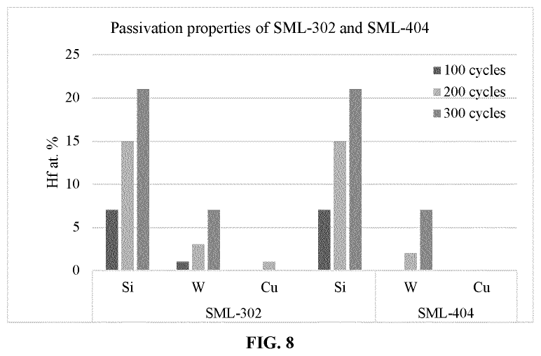

FIG. 8 Passivation properties against ALD of HfOx at 200° C.

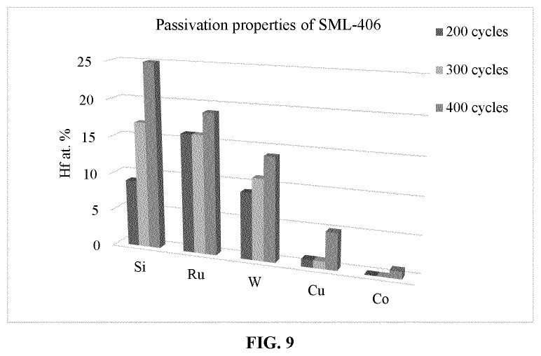

FIG. 9 Passivation properties against ALD of HfOx at 200° C.

### SUMMARY

This disclosure describes spin coatable compositions comprising a liquid crystal with an anchoring group and an organic spin coating solvent, which forms, upon spin coating on an IC substrate, containing metallic surface areas and non-metallic inorganic silicon compound surface areas, a self-assembled monolayer (SAM) selectively on some of these areas, modifying the properties of these areas. Examples of non-metallic inorganic silicon areas are silicon oxide (SiO₂), Silicon Nitride (SiN), and Silicon oxynitride, while examples of metallic areas are copper and tungsten. Selective SAM monolayers on either of these surface area substrate types depend on the anchoring group and the compound (LCC) with different anchoring groups by spin coating and baking and washing this film with treating solutions (FIG. 1). This type of selective surface property enhancement enables selective passivation for atomic layer deposition of dielectric metal oxides such as Titanium oxide, aluminum oxide, hafnium oxide and the like.

More specifically, this invention pertains to a template which is a patterned liquid crystal monolayer structure (100) on a patterned substrate (900), comprising:
the patterned substrate (900) comprising a first surface area (500) having a liquid crystal compound (300) adsorbed thereon as a self-assembled monolayer (SAM) (200) and a second surface area (400) free of the liquid crystal compound (300);
   wherein said liquid crystal compound (300) consists of a cylindrical linear organic liquid crystal core structure comprising at least one 1,4-phenylene moiety (700) to which is attached at one end a linear alkyl group comprising at least two carbons (600) and at the other end a polar anchor group (800) selected from the group consisting of a phosphonate ester comprising moiety, a phosphonic acid comprising moiety, a thiol comprising moiety, an amino comprising moiety, at least one alkylenehydroxy comprising moiety, and an alkyl-polyol comprising moiety;
   wherein in said self-assembled monolayer (200), said liquid crystal compound (300) is arranged in the self-assembled monolayer (200) so that each liquid crystal compounds in said self-assembled monolayer (200) are parallel to each other and pointing in the same direction, perpendicular to said patterned substrate (900) and attached to surface area (500) only through said polar anchor group (800); and
      wherein either,
         when said polar anchor group (800) is either an alkyl-polyol coordination moiety or at least one alkylenehydroxy comprising moiety, said surface area (500) to which said self-assembled monolayer (200) is attached, is a non-metallic inorganic silicon compound-based surface area and said surface area (400) free of the liquid crystal compound (300) is a metallic surface area or,
         when said polar anchor group is selected from the group consisting of a phosphonate ester comprising moiety, a phosphonic acid comprising moiety, a thiol comprising moiety, and an amino comprising moiety, said surface area (500) to which said self-assembled monolayer (200) is attached, is a metallic surface area and said surface area (400) free of the liquid crystal compound (300) is a non-metallic inorganic silicon compound-based surface area.

FIG. 2 shows a schematic depiction of a portion of a cross-section taken perpendicularly to the above-described template and shows captions illustrating the above-described portions of the above-described template. This template on a substrate is made up of a protective SAM of a liquid crystal which has been selectively formed only in certain areas of this substrate to enhance atomic layer deposition (ALD) of a dielectric metal oxide in those regions not covered by this SAM.

This invention also pertains to a composition comprising a liquid crystal compound (300) and an organic spin casting solvent, wherein said liquid crystal compound consists of a cylindrical linear organic liquid crystal core structure comprising at least one 1,4-phenylene moieties (700) to which is attached at one end a linear alkyl group with at least two carbons (600) and at the other end a polar anchor group (800). FIG. 3 shows a schematic depiction of this liquid crystal compound (300).

This invention also pertains to a process for preparing the above-described template and using this template to affect selective ALD of a dielectric metal oxide on the areas of the template not protected by the SAM of liquid crystal (300). Another aspect of the present invention is the use of the composition according to any one of claims 10 to 27 or the liquid crystal compound (300) as defined in any one of claims 1 to 9 for selectively forming a self-assembled monolayer on either metallic or non-metallic areas of a mixed substrate which comprises both metallic and nonmetallic areas.

### DETAILED DESCRIPTION

It is to be understood that both the foregoing general description and the following detailed description are illustrative and explanatory and are not restrictive of the subject matter as claimed. In this application, the use of the singular includes the plural, the word “a” or “an” means “at least one,” and the use of “or” means “and/or,” unless specifically stated otherwise. Furthermore, the use of the term “including,” as well as other forms such as “includes” and “included,” is not limiting. Also, terms such as “element” or “component” encompass both elements and components comprising one unit and elements or components that comprise more than one unit, unless specifically stated otherwise. As used herein, the conjunction “and” is intended to be inclusive and the conjunction “or” is not intended to be exclusive unless otherwise indicated. For example, the phrase “or, alternatively” is intended to be exclusive. As used herein, the term “and/or” refers to any combination of the foregoing elements including using a single element.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature references and similar materials defines a term in a manner that contradicts the definition of that term in this application, this application controls.

Unless otherwise indicated, “alkyl” refers to hydrocarbon groups which can be linear, branched (e.g., methyl, ethyl, propyl, isopropyl, tert-butyl and the like) or cyclic (e.g., cyclohexyl, cyclopropyl, cyclopentyl and the like) multicyclic (e.g., norbornyl, adamantyl and the like). These alkyl moieties may be substituted or unsubstituted as described below. The term “alkyl” refers to such moieties with C-1 to C-8 carbons. It is understood that for structural reasons linear alkyls start with C-1, while branched alkyls and cyclic alkyls start with C-3 and multicyclic alkyls start with C-5. Moreover, it is further understood that moieties derived from alkyls described below, such as alkyloxy (alkoxy), have the same carbon number ranges unless otherwise indicated. The same criteria apply to the designation C-1 to C-4 alkyl. If the length of the alkyl group is specified as other than described above, the above-described definition of alkyl still stands with respect to it encompassing all types of alkyl moieties as described above and that the structural consideration with regards to minimum number of carbons for a given type of alkyl group still apply.

Alkyloxy (a.k.a. Alkoxy) refers to an alkyl group on which is attached through an oxy (—O—) moiety (e.g., methoxy, ethoxy, propoxy, butoxy, 1,2-isopropoxy, cyclopentyloxy cyclohexyloxy and the like). These alkyloxy moieties may be substituted or unsubstituted as described below. The criteria for establishing the nature of the alkyl in C-1 to C-8 alkoxy or C-1 to C-4 alkoxy are the same as previously described for alkyl moieties.

Halo or halide refers to a halogen, F, Cl, Br or I which is linked by one bond to an organic moiety.

Haloalkyl refers to a linear, cyclic or branched saturated alkyl group such as defined above in which at least one of the hydrogens has been replaced by a halide selected from the group of F, Cl, Br, I or mixture of these if more than one halo moiety is present. Fluoroalkyls are a specific subgroup of these moieties.

The term “alkylene”, unless otherwise indicated refers to hydrocarbon groups which can be a linear, branched or cyclic which has two or more attachment points (e.g., of two attachment points: methylene, ethylene, 1,2-isopropylene, a 1,4-cyclohexylene and the like; of three attachment points 1,1,1-substituted methane, 1,1,2-substituted ethane, 1,2,4-substituted cyclohexane and the like). Here again, when designating a possible range of carbons, such as C-1 to C-20, as a non-limiting example, this range encompasses linear alkylenes starting with C-1 but only designates branched alkylenes, or cycloalkylene starting with C-3. These alkylene moieties may be substituted or unsubstituted as described below. The term linear alkylene, refers to a linear alkylene moiety with two attachment points, which is unsubstituted, unless otherwise indicated.

The term “acyl” refers to a (—C═O)—R moiety where R is H, an aryl or an alkyl.

The term “aryl” or “aromatic groups” refers to such groups which contain 6 to 24 carbon atoms including phenyl, tolyl, xylyl, naphthyl, anthracyl, biphenyls, bis-phenyls, tris-phenyls and the like. These aryl groups may further be substituted with any of the appropriate substituents, e.g., alkyl, alkoxy, acyl or aryl groups mentioned hereinabove.

Unless otherwise indicated in the text, the term “substituted” when referring to an aryl, alkyl, alkyloxy, fluoroalkyl, fluoroalkyloxy, fused aromatic ring, arene, heteroarene refers to one of these moieties which also contain with one or more substituents, selected from the group of unsubstituted alkyl, substituted alkyl, unsubstituted aryl, alkyloxyaryl (alkyl-O-aryl-), dialkyloxyaryl ((alkyl-O—) 2-aryl), haloaryl, alkyloxy, alkylaryl, haloalkyl, halide, hydroxyl, cyano, nitro, acetyl, alkylcarbonyl, formyl, ethenyl (CH₂═CH—), phenylethenyl (Ph-CH═CH—), arylethenyl (Aryl-CH═CH), and substituents comprising ethenylenearylene moieties (e.g., Ar(—CH═CH—Ar—)_{z} where z is 1-3. Specific, non-limiting examples of substituted aryl and substituted aryl ethenyl substituent are as follows where “ [symbol] ” represents the point of attachment:

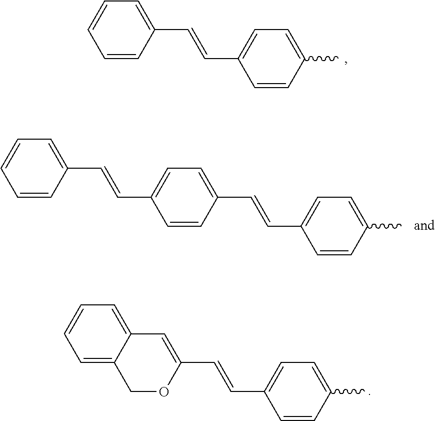

The term amino comprising moiety when used in the context of the polar anchoring group (shown schematically as (800) in FIG. 3) of the liquid crystal molecules used in the inventive SAM template (FIG. 2) and in the inventive liquid crystal composition, refers to an amino group (—NH₂), which can be directly bond to the liquid crystal molecule through a covalent bond or through a C-1 to C-8 linear alkylene moiety (-alkylene-NH₂) or a C-2 to C-8 linear oxyalkylene moiety (—O-alkylene-NH₂).

The term phosphonate ester comprising moiety when used in the context of the polar anchoring group (shown schematically as (800) in FIG. 3) of the liquid crystal molecules used in the inventive SAM template (FIG. 2) and in the inventive liquid crystal composition refers to a dialkyl phosphonate group (—P(═O)(O-alkyl)₂), moiety, which can be directly bound to the liquid crystal molecule through a covalent bond or through a C-1 to C-8 linear alkylene moiety (-alkylene-P(═O)(O-alkyl)₂) or a C-2 to C-8 linear oxyalkylene moiety (—O-alkylene-P(═O)(O-alkyl)₂).

The term phosphonic acid moiety when used in the context of the polar anchoring group (shown schematically as (800) in FIG. 3) of the liquid crystal molecules used in the inventive SAM template (FIG. 2) and in the inventive liquid crystal composition, refers to a phosphonic acid group (—P(═O)(OH)₂), moiety, which can be directly bound to the liquid crystal molecule through an a covalent bond or through a C-1 to C-8 linear alkylene moiety (-alkylene-P(═O)(OH)₂) or a C-1 to C-8 linear oxyalkylene moiety (—O-alkylene-P(═O)(OH)₂).

The term a thiol comprising moiety when used in the context of the polar anchoring group (shown schematically as (800) in FIG. 3) of the liquid crystal molecules used in the inventive SAM template (FIG. 2) and in the inventive liquid crystal composition, refers to a thiol group (—SH), moiety, which is bound to the liquid crystal molecule through C-1 to C-8 linear alkylene moiety (-alkylene-SH) or a C-2 to C-8 linear oxyalkylene moiety (—O-alkylene-SH).

The term alkylenehydroxy comprising moiety when used in the context of the polar anchoring group (shown schematically as (800) in FIG. 3) of the liquid crystal molecules used in the inventive SAM template and in the inventive liquid crystal composition, refers to a hydroxy group (—OH), moiety, which is bound to the liquid crystal molecule through either a C-1 to C-8 linear oxy-alkylene moiety (—O-alkylene-OH) or a C-1 to C-8 linear alkylene moiety (-alkylene-OH).

The term alkyl-polyol comprising moiety when used in the context of the polar anchoring group (shown schematically as (800) in FIG. 3) of the liquid crystal molecules used in the inventive SAM template and in the inventive liquid crystal composition, is an alkyl polyol (—C(alkylene-OH)ₙ, where n is 2 or 3) which is which is bound to the liquid crystal molecule through C-2 to C-8 linear alkylene moiety (-alkylene-C(alkylene-OH)ₙ, where n is 2 or 3) or a C-2 to C-8 linear oxyalkylene moiety (—O-alkylene-C(alkylene-OH)ₙ, where n is 2 or 3)).

The term “cylindrical linear organic liquid crystal core structure comprising at least one 1,4-phenylene moiety,” (as shown schematically in FIG. 3 as (700)) is a moiety which comprises at least one 1,4-phenylene moiety, but which may also comprise additional different linear moieties such as additional 1,4-phenylene moieties which may be either substituted or unsubstituted, a 1,4-cyclohexylene moiety, a 1,4-phenylene, a linear ethylene moiety, a trans ethenylene moiety

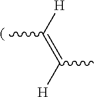

where [symbol] represents the point of attachment on the C(H)═C(H) moiety), where each of these linear moieties are attached to each other forming a linear arrangement in a liquid crystal molecule which facilitates the pi-pi interaction of one or more of the 1,4-phenylene moiety in different liquid crystal molecules where said liquid crystal molecules (300), (consisting of said linear alkyl group (600), said cylindrical linear organic liquid crystal core structure (700) and said polar anchor group (800)) interact with each other through these pi-pi interactions when these are grafted to specific areas of a patterned substrate through their polar anchor group (800) (FIG. 2). Non limiting examples of cylindrical linear organic liquid crystal core structure comprising at least one 1,4-phenylene moieties, are shown in structures (I) to (VIII), as described herein.

The term “linear alkyl group comprising at least two carbons,” (as shown schematically in FIG. 3 as (600)) of the liquid crystal molecules used in the inventive SAM template (FIG. 2) and in the inventive liquid crystal composition is a linear alkyl (a.k.a. n-alkyl moiety), which has at least two carbon atoms and is unsubstituted.

One aspect of this invention pertains to an inventive template which is a patterned liquid crystal monolayer structure (100) on a patterned substrate (900), comprising:
the patterned substrate (900) comprising a first surface area (500) having a liquid crystal compound (300) adsorbed thereon as a self-assembled monolayer (SAM) (200) and a second surface area (400) free of the liquid crystal compound (300);
   wherein said liquid crystal compound (300) consists of a cylindrical linear organic liquid crystal core structure comprising at least one 1,4-phenylene moiety (700) to which is attached at one end a linear alkyl group comprising at least two carbons (600) and at the other end a polar anchor group (800) selected from the group consisting of a phosphonate ester comprising moiety, a phosphonic acid comprising moiety, a thiol comprising moiety, an amino comprising moiety, at least one alkylenehydroxy comprising moiety, and an alkyl-polyol comprising moiety;
   wherein in said self-assembled monolayer (200), said liquid crystal compound (300) is arranged in the self-assembled monolayer (200) so that each liquid crystal compounds in said self-assembled monolayer (200) are parallel to each other and, pointing in the same direction, perpendicular to said patterned substrate (900) and attached to surface area (500) only through said polar anchor group (800); and
      wherein either,
         when said polar anchor group (800) is an alkyl-polyol coordination moiety or at least one alkylenehydroxy comprising moiety, said surface area (500), to which said self-assembled monolayer (200) is attached, is a non-metallic inorganic silicon compound-based surface area and said surface area (400) free of the liquid crystal compound (300) is a metallic surface area or,
         when said polar anchor group is selected from the group consisting of a phosphonate ester comprising moiety, a phosphonic acid comprising moiety, a thiol comprising moiety, and an amino comprising moiety, said surface area (500), to which said self-assembled monolayer (200) is attached, is a metallic surface area and said surface area (400) free of the liquid crystal compound (300) is a non-metallic inorganic silicon compound-based surface area.

FIG. 2 shows a schematic depiction of a portion of a cross section taken perpendicularly to the above-described template and captions illustrative the above-described portions the above described template on a substrate on which a protective SAM of a liquid crystal has been selectively formed only in certain areas of this substrate. This selective formation of a SAM acts to enhance atomic layer deposition (ALD) of a dielectric metal oxide in those regions not covered by this SAM.

Another aspect of this inventive template is when it is a template consisting of the elements (100), (200), (300), (400), (500), (600), (700), and (800), as described above, and no other elements.

Another aspect of this inventive template is when said linear alkyl group (600) is a C-2 to C-5 alkyl. In one aspect of this embodiment the linear alkyl group is ethyl, in another aspect it is n-propyl, in another aspect it is n-butyl, and in another aspect, it is n-pentyl.

In another aspect of this inventive template, as described herein, said cylindrical linear organic liquid crystal core structure is one comprising at least two 1,4-phenylene moieties (700), selected from the group consisting of structures (I), (II), (IIa), (III), (IV), (V), (VI), (VII), and (VIII), wherein ** is the attachment point of said linear alkyl group (600) and * is the attachment point of said polar anchor group (800), R₁, and R₂ are individually selected from H, a C-1 to C-2 alkyl and F. In another aspect of this embodiment said 1,4-phenylene moieties (700) has structure (I), in another aspect it has structure (II), in another aspect it has structure (IIa), in another aspect it has structure (III), in another aspect it has structure (IV), in another aspect it has structure (V), in another aspect it has structure (VI), in another aspect it has structure (VII), in a final embodiment it has structure (VIII).

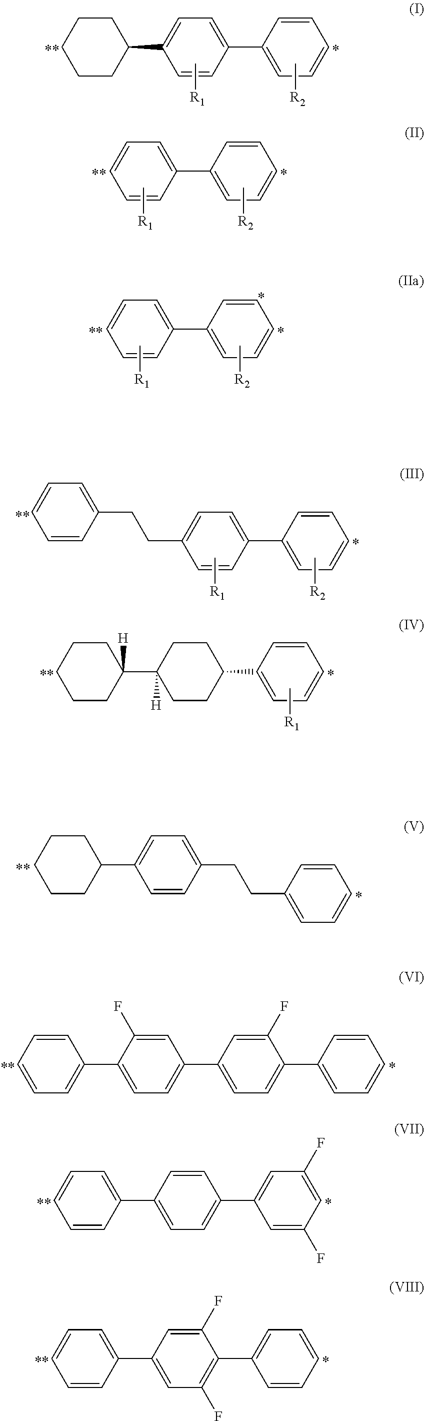

In another aspect of this inventive template, as described herein, said polar grafting (a.k.a. anchor) group (800) is selected from the group consisting of structure (Ip), structure (Ipa), structure (IIp), and structure (IIpa), wherein L₁, and L₂ are individually selected from a C-2 to C-4 linear alkylene spacer, and *** is the attachment point of said polar anchor group to said liquid crystal compound. In another aspect of this embodiment said polar grafting group (800) has structure (Ip), in another it has structure (Ipa) in another it has structure (IIp), in another it has structure (IIpa). In one aspect of the embodiment with structure (Ip), L₁ is a C-2 linear alkylene group, in another it is a C-3 linear group, in another it is a C-4 linear alkylene group. In one aspect of the embodiment with structure (Ipa), L₁ is a C-2 linear alkylene group, in another it is a C-3 linear group, in another it is a C-4 linear alkylene group. In one aspect of the embodiment with structure (IIp), L₂ is a C-2 linear alkylene group, in another it is a C-3 linear group, in another it is a C-4 linear alkylene group. In one aspect of the embodiment with structure (IIpa), L₂ is a C-2 linear alkylene group, in another it is a C-3 linear group, in another it is a C-4 linear alkylene group.

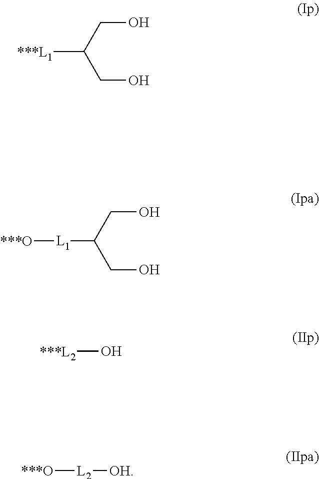

In another aspect of this inventive template, as described herein, said polar grafting group (800) is selected from the group consisting of structure (IIIp), structure (IVp), structure (Vp), structure (VIp), structure (VIIp), structure (VIIIp) and structure (IXp), wherein L₃, L₄, L₅, L₆, and L₇ are individually selected from a C-2 to C-4 linear alkylene spacer, *** is the attachment point of said polar anchor group to said liquid crystal compound, Rₚ₁ is a C-1 to C-4 alkoxy, and Rₚ₂ is a C-1 to C-4 alkyl or a C-1 to C-4 alkoxy. In one aspect of this embodiment said polar grafting group has structure (IIIp), in another aspect of this embodiment L₃ is a C-2 linear alkylene, in another L₃ is a C-3 linear alkylene, in another is it a C-4 linear alkylene. In one aspect of this embodiment said polar grafting group has structure (IVp), In one aspect of this embodiment L₄ is a C-2 linear alkylene, in another L₄ is a C-3 linear alkylene, in another is it a C-4 linear alkylene. In one aspect of this embodiment said polar grafting group has structure (Vp), in one aspect of this embodiment Rₚ₁ is a methoxy, in another it is ethoxy, in another it is n-propoxy, in another it is n-butoxy; in one aspect of this embodiment Rₚ₂ is a methyl, in another it is ethyl, in another it is n-propyl, in another it is n-butyl; in another aspect of this embodiment Rₚ₂ is a methoxy, in another it is ethoxy, in another it is n-propoxy, in another it is n-butoxy. In one aspect of this embodiment said polar grafting group has structure (VIp), in one aspect of this embodiment Rₚ₁ is a methoxy, in another it is ethoxy, in another it is n-propoxy, in another it is n-butoxy; in one aspect of this embodiment Rₚ₂ is a methyl, in another it is ethyl, in another it is n-propyl, in another it is n-butyl; in another aspect of this embodiment Rₚ₂ is a methoxy, in another it is ethoxy, in another it is n-propoxy, in another it is n-butoxy. In another aspect of this embodiment said polar grafting group has structure (VIIp). In another aspect of this embodiment said polar grafting group has structure (VIIIp), in one aspect of this embodiment L₆ is a C-2 linear alkylene, in another aspect it is a C-3 linear alkylene, in another aspect it is a C-4 linear alkylene. In another aspect of this embodiment said polar grafting group has structure (IXp), in one aspect of this embodiment L₇ is a C-2 linear alkylene, in another aspect it is a C-3 linear alkylene, in another aspect it is a C-4 linear alkylene.

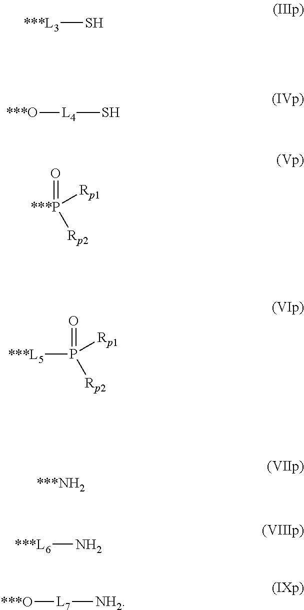

In another aspect of this inventive template, as described herein, said liquid crystal compound (300) is one selected from group consisting of one having structures (M1), (M2), (M3), (M4), (M5), and (M7). In one aspect of this embodiment said liquid crystal compound (300) has structure (M1), in another it has structure (M2), in another it has structure (M3), in another it has structure (M4), in another it has structure (M5), in another it has structure (M7).

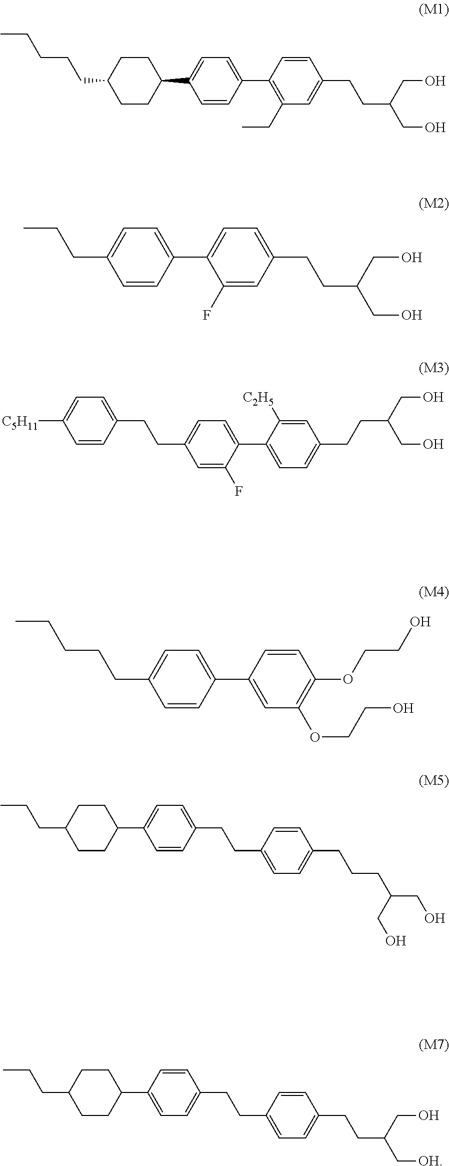

In another aspect of this inventive template, as described herein, said liquid crystal compound (300) is one selected from group consisting of one having structures (M8), (M9), (M10), (M11), (M12), (M13) and (M14). In one aspect of this embodiment said liquid crystal compound (300) has structure (M8), in another it has structure (M9), in another it has structure (M10), in another it has structure (M11), in another it has structure (M12), in another it has structure (M13), in another it has structure (M14).

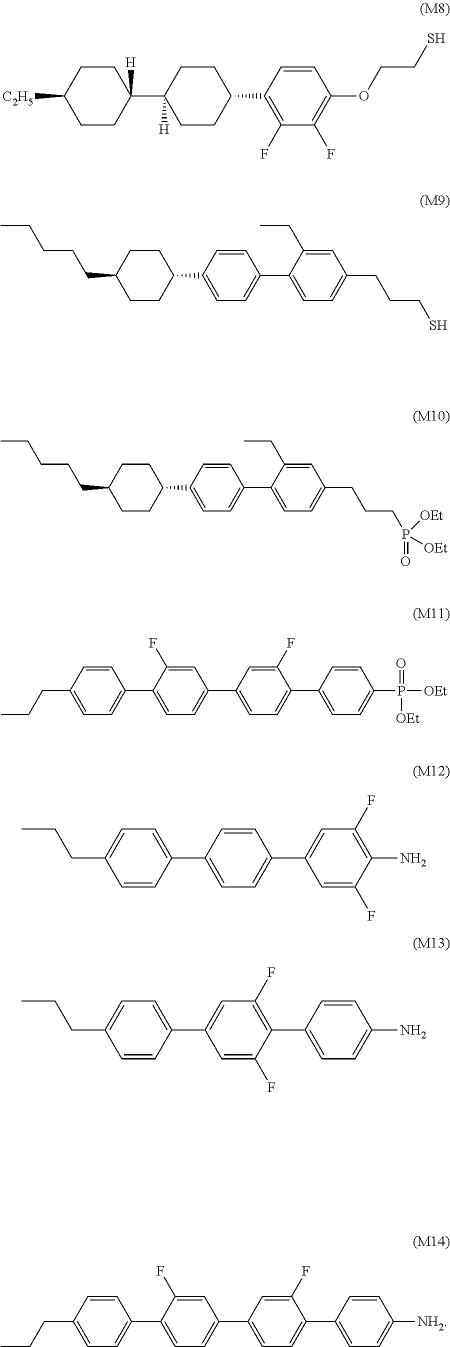

In another aspect of this inventive template, as described herein, where said surface area (500) overlaid with a SAM is a non-metallic inorganic silicon compound-based surface area selected from the group consisting of silicon oxide (SiO₂), silicon with a native oxide, silicon nitride (SiN) and silicon oxynitride (SiON) and said surface area which is bare (400), is a metal which is selected from the group consisting of tungsten, gold, silver, copper, cobalt, ruthenium, zirconium, titanium, and hafnium. In one aspect of this embodiment said surface area (500) overlaid with a SAM is a non-metallic inorganic silicon based surface areas is silicon dioxide (SiO₂), in another it is Silicon (Si) with a native oxide, in another it is silicon nitride (SiN), in another it is silicon oxynitride (SiON); in these aspects, in one aspect, said surface area which is bare (400) is tungsten, in another it is gold, in another it is silver, in another it is copper, in another it is cobalt, in another it is ruthenium, in another it is zirconium, in another it is titanium, in another it is hafnium.

In another aspect of this inventive template, as described herein, where said surface area (500) overlaid with a SAM is a metal selected from the group consisting of tungsten, gold, silver, copper, cobalt, ruthenium, zirconium, titanium, and hafnium and said surface area which is bare (400), is a non-metallic inorganic silicon compound-based surface area which is selected from the group the group consisting of silicon oxide (SiO₂), silicon with a native oxide, silicon nitride (SiN) and silicon oxynitride (SiON). In one aspect of this embodiment said surface area (500) overlaid with a SAM is tungsten, in another it is gold, in another it is silver, in another it is copper, in another it is cobalt, in another it is ruthenium, in another it is zirconium, in another it is titanium, in another it is hafnium; in these aspects, in one aspect, said surface area which is bare (400) is silicon dioxide (SiO₂), in another it is Silicon (Si) with a native oxide, in another it is silicon nitride (SiN), in another it is silicon oxynitride (SiON).

### Inventive Compositions

Another aspect of this invention is a composition comprising a liquid crystal compound (300) and an organic spin casting solvent, wherein said liquid crystal compound consists of a cylindrical linear organic liquid crystal core structure comprising at least one 1,4-phenylene moieties (700) to which is attached at one end a linear alkyl group with at least two carbons (600) and at the other end a polar anchor group (800). In one aspect of this embodiment said composition consists of a liquid crystal compounds (300) and an organic spin casting solvent.

In another aspect of the above-described composition in liquid crystal compound (300) said linear alkyl group (600) is a C-2 to C-5 alkyl. In one aspect of this embodiment said linear alkyl group is ethyl, in another it is n-propyl, in another it is n-butyl.

In another aspect of the above-described composition in liquid crystal compound (300) said cylindrical linear organic liquid crystal core structure comprising at least two 1,4-phenylene moieties (700) is selected from the group consisting of structures (I), (II), (IIa), (III), (IV), (V), (VI), (VII), and (VIII), wherein ** is the attachment point of said linear alkyl group (600) and * is the attachment point of said polar anchor group (800), R₁, and R₂ are individually selected from H, a C-1 to C-2 alkyl and F. In another aspect of this embodiment said linear organic liquid crystal core structure comprising at least two 1,4-phenylene moieties (700) has structure (I); in one aspect of this embodiment R₁ is H, in another it is methyl, in another it is F; in one aspect of these embodiments R₂ is H, in another it is methyl, in another it is F. In another aspect of this embodiment said linear organic liquid crystal core structure comprising at least two 1,4-phenylene moieties (700) has structure (II); in one aspect of this embodiment R₁ is H, in another it is methyl, in another it is F; in one aspect of these embodiments R₂ is H, in another it is methyl, in another it is F. In another aspect of this embodiment said linear organic liquid crystal core structure comprising at least two 1,4-phenylene moieties (700) has structure (IIa); in one aspect of this embodiment R₁ is H, in another it is methyl, in another it is F; in one aspect of these embodiments R₂ is H, in another it is methyl, in another it is F. In another aspect of this embodiment said linear organic liquid crystal core structure comprising at least two 1,4-phenylene moieties (700) has structure (III); in one aspect of this embodiment R₁ is H, in another it is methyl, in another it is F; in one aspect of these embodiments R₂ is H, in another it is methyl, in another it is F. In another aspect of this embodiment said linear organic liquid crystal core structure comprising at least two 1,4-phenylene moieties (700) has structure (IV); in one aspect of this embodiment R₁ is H, in another aspect it is methyl, and, in another aspect, it is F. In another aspect of this embodiment said linear organic liquid crystal core structure comprising at least two 1,4-phenylene moieties (700) has structure (V). In another aspect of this embodiment said linear organic liquid crystal core structure comprising at least two 1,4-phenylene moieties (700) has structure (VI). In another aspect of this embodiment said linear organic liquid crystal core structure comprising at least two 1,4-phenylene moieties (700) has structure (VI). In another aspect of this embodiment said linear organic liquid crystal core structure comprising at least two 1,4-phenylene moieties (700) has structure (VII). In another aspect of this embodiment said linear organic liquid crystal core structure comprising at least two 1,4-phenylene moieties (700) has structure (VIII).

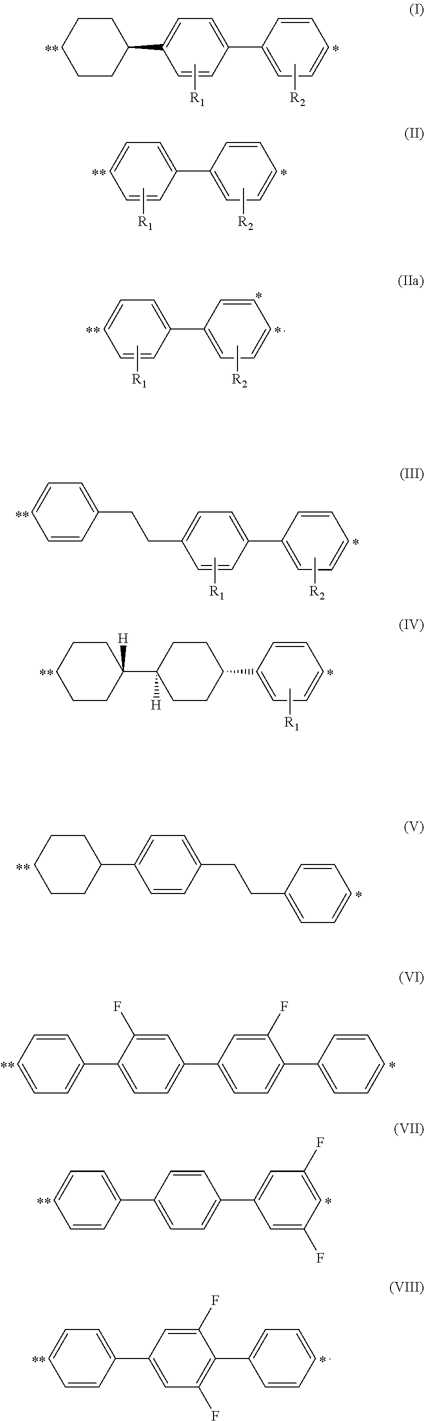

In another aspect of the above-described composition in liquid crystal compound (300) said polar grafting group (800) is selected from the group consisting of structure (Ip), structure (Ipa), structure (IIp), and structure (IIpa), wherein L₁, and L₂ are individually selected from a C-2 to C-4 linear alkylene spacer, and *** is the attachment point of said polar anchor group to said liquid crystal compound. In another aspect of this embodiment said polar grafting group (800) has structure (Ip), in another it has structure (Ipa) in another it has structure (IIp), in another it has structure (IIpa). In one aspect of the embodiment with structure (Ip), L₁ is a C-2 linear alkylene group, in another it is a C-3 linear group, in another it is a C-4 linear alkylene group. In one aspect of the embodiment with structure (Ipa), L₁ is a C-2 linear alkylene group, in another it is a C-3 linear group, in another it is a C-4 linear alkylene group. In one aspect of the embodiment with structure (IIp), L₂ is a C-2 linear alkylene group, in another it is a C-3 linear group, in another it is a C-4 linear alkylene group. In one aspect of the embodiment with structure (IIpa), L₂ is a C-2 linear alkylene group, in another it is a C-3 linear group, in another it is a C-4 linear alkylene group.

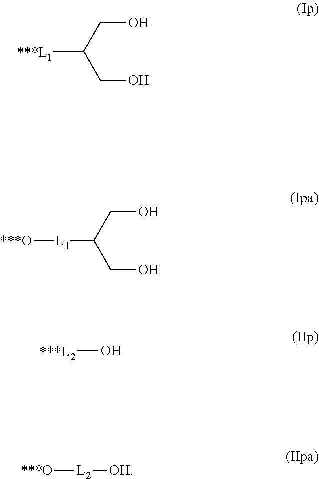

In another aspect of the above-described composition in liquid crystal compound (300) said polar grafting group (800) is selected from the group consisting of structure (IIIp), structure (IVp), structure (Vp), structure (VIp), structure (VIIp), structure (VIIIp) and structure (IXp), wherein L₃, L₄, L₅ L₆ and L₇ are individually selected from a C-2 to C-4 linear alkylene spacer, *** is the attachment point of said polar anchor group to said liquid crystal compound, Rₚ₁ is a C-1 to C-4 alkoxy, and Rₚ₂ is a C-1 to C-4 alkyl or a C-1 to C-4 alkoxy. In one aspect of this embodiment said polar grafting group has structure (IIIp), in another aspect of this embodiment L₃ is a C-2 linear alkylene, in another L₃ is a C-3 linear alkylene, in another is it a C-4 linear alkylene. In one aspect of this embodiment said polar grafting group has structure (IVp), In one aspect of this embodiment L₄ is a C-2 linear alkylene, in another L₄ is a C-3 linear alkylene, in another is it a C-4 linear alkylene. In one aspect of this embodiment said polar grafting group has structure (Vp), in one aspect of this embodiment Rₚ₁ is a methoxy, in another it is ethoxy, in another it is n-propoxy, in another it is n-butoxy; in one aspect of this embodiment Rₚ₂ is a methyl, in another it is ethyl, in another it is n-propyl, in another it is n-butyl; in another aspect of this embodiment Rₚ₂ is a methoxy, in another it is ethoxy, in another it is n-propoxy, in another it is n-butoxy. In one aspect of this embodiment said polar grafting group has structure (VIp), in one aspect of this embodiment Rₚ₁ is a methoxy, in another it is ethoxy, in another it is n-propoxy, in another it is n-butoxy; in one aspect of this embodiment Rₚ₂ is a methyl, in another it is ethyl, in another it is n-propyl, in another it is n-butyl; in another aspect of this embodiment Rₚ₂ is a methoxy, in another it is ethoxy, in another it is n-propoxy, in another it is n-butoxy. In another aspect of this embodiment said polar grafting group has structure (VIIp). In another aspect of this embodiment said polar grafting group has structure (VIIIp), in one aspect of this embodiment L₆ is a C-2 linear alkylene, in another aspect it is a C-3 linear alkylene, in another aspect it is a C-4 linear alkylene. In another aspect of this embodiment said polar grafting group has structure (IXp), in one aspect of this embodiment L₇ is a C-2 linear alkylene, in another aspect it is a C-3 linear alkylene, in another aspect it is a C-4 linear alkylene.

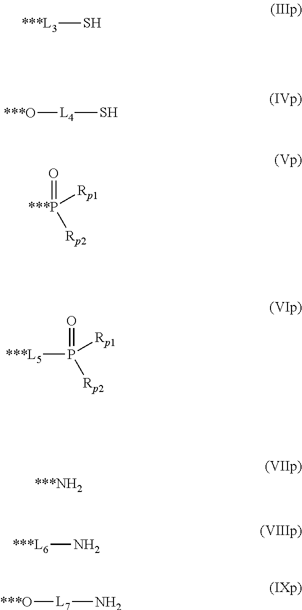

In another aspect of the above-described composition said liquid crystal compound (300) is selected from the group consisting of one having structures (M1), (M2), (M3), (M4), (M5), and (M7). In one aspect of this embodiment, it has structure (M1), in another it has structure (M2), in another it has structure (M3), in another it has structure (M4), in another it has structure (M5), in another it has structure (M7).

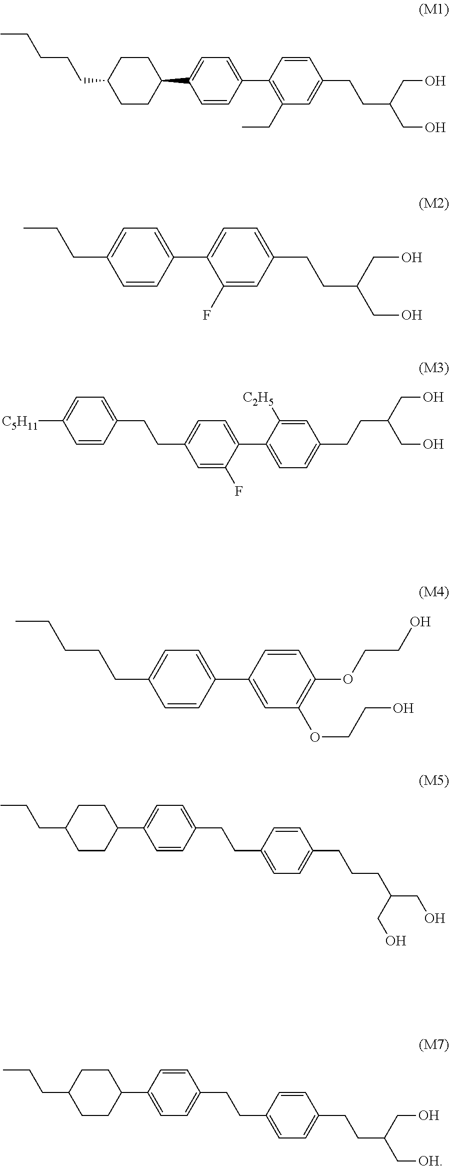

In another aspect of the above-described composition said liquid crystal compound (300) is selected from the group consisting of one having structures (M8), (M9), (M10), (M11), (M12), (M13) and (M14). In one aspect of this embodiment, it has structure (M8), in another it has structure (M10), in another it has structure (M11), in another it has structure (M12), in another it has structure (M13), in another it has structure (M14).

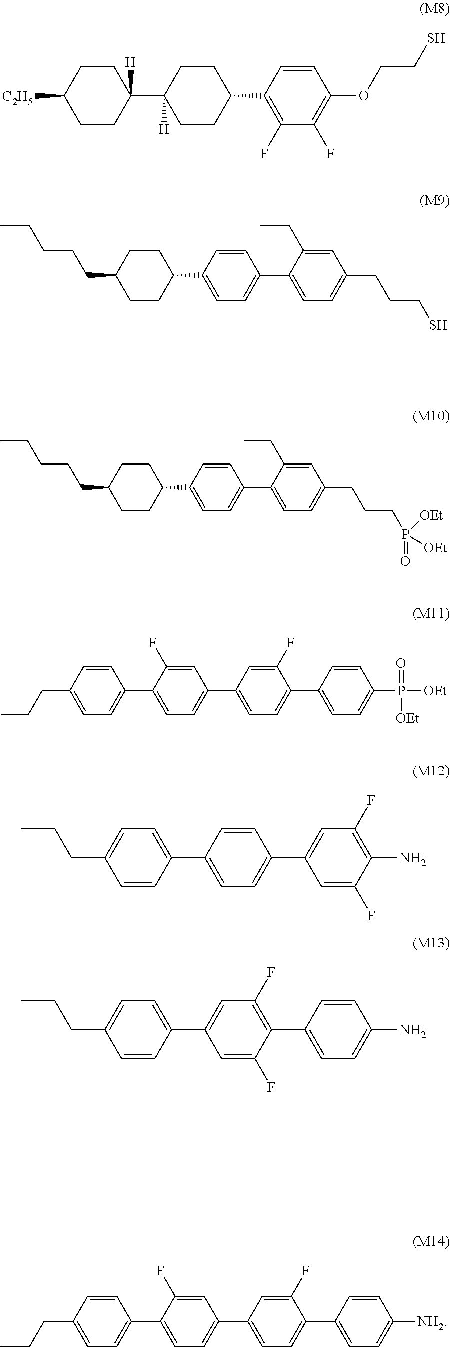

In another aspect of the above-described composition said liquid crystal compound (300) is one having structure (M4),

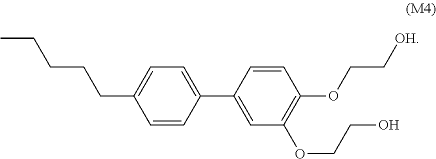 [SMILES: CCCCCc1ccc(-c2ccc(OCCO)c(OCCO)c2)cc1]

In another aspect of the above-described composition said liquid crystal compound (300) is one selected from the group consisting of one having structures (M8), (M9), (M12), (M13) and (M14). In one aspect of this embodiment, it has structure (M8), in another aspect it has structure (M9), in another aspect it has structure (M12), in another aspect it has structure (M13), in another aspect it has structure (M14).

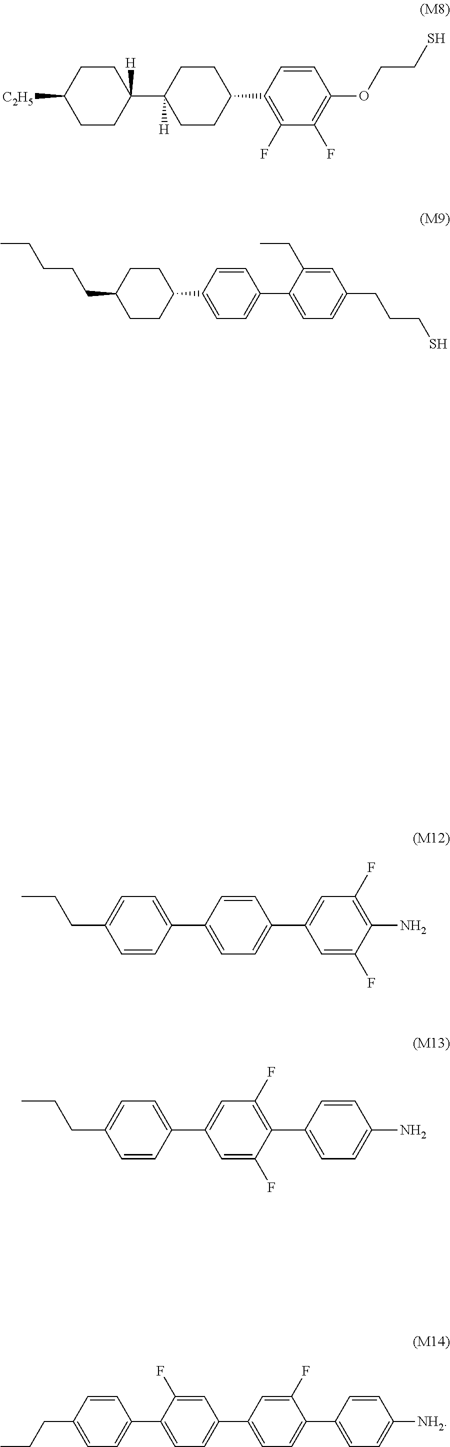 [SMILES: CC[C@H]1CC[C@H]([C@H]2CC[C@H](c3ccc(OCCS)c(F)c3F)CC2)CC1 ; CCCCC[C@H]1CC[C@H](c2ccc(-c3ccc(CCCS)cc3CC)cc2)CC1 ; CCCc1ccc(-c2ccc(-c3cc(F)c(N)c(F)c3)cc2)cc1 ; CCCc1ccc(-c2cc(F)c(-c3ccc(N)cc3)c(F)c2)cc1 ; CCCc1ccc(-c2ccc(-c3ccc(-c4ccc(N)cc4)c(F)c3)cc2F)cc1]

In another aspect of the above-described composition said liquid crystal compound (300) is one selected from the group consisting of one having structures (M10), and (M11). In one aspect of this embodiment, it has structure (M10), in another it has structure (M11).

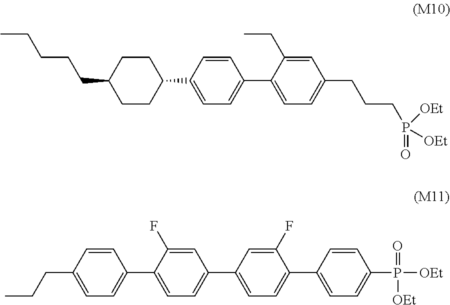 [SMILES: CCCCC[C@H]1CC[C@H](c2ccc(-c3ccc(CCCP(=O)(OCC)OCC)cc3CC)cc2)CC1 ; CCCc1ccc(-c2ccc(-c3ccc(-c4ccc(P(=O)(OCC)OCC)cc4)c(F)c3)cc2F)cc1]

In another aspect of the above-described said liquid crystal compound is present at a loading of about 0.5 wt. % to about 2.0 wt. % in said organic spin coating solvent.

In another aspect of the above-described composition said organic spin coating solvent is a single organic solvent or a mixture of at least two organic solvents selected from the group consisting of a glycol ether derivative selected from ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA), a carboxylate of a mono-basic acid selected from ethyl acetate, n-butyl acetate and amyl acetate, a carboxylate of di-basic acids selected from diethyloxylate and diethylmalonate, a dicarboxylates of a glycol selected from ethylene glycol diacetate and propylene glycol diacetate, a hydroxy carboxylate selected from methyl lactate, ethyl lactate (EL), ethyl glycolate, and ethyl-3-hydroxy propionate, a ketone ester selected from methyl pyruvate and ethyl pyruvate, an alkyloxycarboxylic acid ester selected from methyl 3-methoxypropionate, ethyl 3-ethoxypropionate, ethyl 2-hydroxy-2-methylpropionate, and methylethoxypropionate, a ketone derivative selected from methyl ethyl ketone, acetyl acetone, cyclopentanone, cyclohexanone and 2-heptanone, diacetone alcohol methyl ether, a ketone alcohol derivative selected from acetol and diacetone alcohol, a ketal or acetal selected from 1,3 dioxalane and diethoxypropane, butyrolactone, an amide selected from dimethylacetamide and dimethylformamide, and anisole.

In another aspect of the above-described compositions said organic spin coating solvent is a mixture of PGME and PGMEA. In one aspect of this embodiment, it is from about 50 wt. % PGME to about 80 wt. % PGMEA and about 20 wt. % PGMEA to about 80 wt. % PGMEA. In one aspect of this embodiment, it is about 70 wt. % PGME and about 30 wt. % PGMEA.

In another aspect of the above-described compositions they can additionally contain surfactants as additives to facilitate coating.

### Methods of Using Inventive Compositions.

Another aspect of this invention is a process for forming a self-assembled monolayer (SAM) of a liquid crystal (LC) selectively on nonmetallic areas in a mixed substrate which comprises both metallic and nonmetallic areas, the process comprising the steps:
i) spin coating on a mixed substrate any one of the compositions as described wherein said liquid crystal has a polar anchor group (800) which is an alkyl-polyol coordination moiety or at least one alkylenehydroxy comprising moiety, as described herein, wherein said nonmetallic areas are selected from silicon dioxide, silicon with native oxide, silicon nitride, and silicon oxynitride, and said metallic areas are selected from the group consisting of tungsten, gold, silver, copper, cobalt, ruthenium, zirconium, titanium, and hafnium,
ii) baking at a temperature ranging from about 150° C. to about 180° C. for about 2 min to about 10 min under an inert gas,
iii) rinsing with an organic spin coating solvent,
iv) air drying substrate,
v) repeat steps i) to iv) two times, to obtain self-assembled monolayer of liquid crystal on both the metallic and non-metallic areas,
vi) rinsing the substrate with a dilute aqueous solution of an acid to selectively remove the self-assembled monolayer of liquid crystal on the metallic areas,
vii) rinsing the substrate with water and air drying to obtain a substrate in where the non-metallic areas only have a SAM of a LC.
“repeat steps i) to iv) two times” in step v) means that the process comprises in total at least three completions of steps i) to iv).

In one aspect of this process said liquid crystal is one which whose polar grafting group is either an alkyl-polyol coordination moiety or at least one alkylenehydroxy comprising moiety a selected from the group consisting of structure (Ip), structure (Ipa), structure (IIp), and structure (IIpa), wherein L₁, and L₂ are individually selected from a C-2 to C-4 linear alkylene spacer, and *** is the attachment point of said polar anchor group to said liquid crystal compound. In another aspect of this embodiment said polar grafting group (800) has structure (Ip), in another it has structure (Ipa) in another it has structure (IIp), in another it has structure (IIpa). In one aspect of the embodiment with structure (Ip), L₁ is a C-2 linear alkylene group, in another it is a C-3 linear group, in another it is a C-4 linear alkylene group. In one aspect of the embodiment with structure (Ipa), L₁ is a C-2 linear alkylene group, in another it is a C-3 linear group, in another it is a C-4 linear alkylene group. In one aspect of the embodiment with structure (IIp), L₂ is a C-2 linear alkylene group, in another it is a C-3 linear group, in another it is a C-4 linear alkylene group. In one aspect of the embodiment with structure (IIpa), L₂ is a C-2 linear alkylene group, in another it is a C-3 linear group, in another it is a C-4 linear alkylene group.

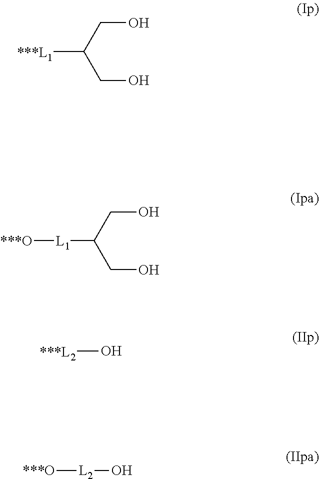

In another aspect of this process said liquid crystal is selected from the group consisting of ones having structures (M1), (M2), (M3), (M4), (M5) and (M7). In a more specific embodiment said liquid crystal has structure (M1), in another it has structure (M2), in another it has structure (M3), in another it has structure (M4), in another it has structure (M5) and in another it has structure (M7).

Another aspect of this invention is a process for selective atomic layer deposition on metallic areas of a mixed substrate comprising both metallic and non-metallic areas, the process comprising the steps:
ia) producing a mixed substrate in which non-metallic areas have a self-assembled monolayer of liquid crystal according to the above-described process which employs any one of compositions as described herein where said liquid crystal has a polar anchor group (800) which is either an alkyl-polyol coordination moiety or at least one alkylenehydroxy,
iia) using an atomic layer deposition technique, deposit a metal oxide more selectively on the metallic areas which do not have a self-assembled monolayer of liquid crystal.

In a more specific aspect of this process, said polar anchor group (800) is selected from the group consisting of structure (Ip), structure (Ipa), structure (IIp) and structure (IIpa) as described herein.

In a more specific aspect of this process said liquid crystal is one having structure (M1), (M3), (M4), (M5) or (M7) as described herein. In one aspect of this embodiment, it has structure (M1). In another aspect of this embodiment, it has structure (M3). In another aspect of this embodiment, it has structure (M4). In another aspect of this embodiment, it has structure (M5). In another aspect of this embodiment, it has structure (M7).

In a more specific embodiment of this process said atomic layer deposition technique is one employing deposition of about 10 to about 50 cycles of deposition, wherein each cycle employs a vapor treatment with (MeCp)₂Hf(OMe)Me for about 1 to about 5 s followed by a treatment with N₂ for about 5 s to about 15 s, a treatment with H₂O for about 1 s to about 5 s, and a treatment with N₂ for about 5 s to about 15 s at a temperature of about 250° C. to about 350° C.

Another aspect of this invention is a process for forming a self-assembled monolayer (SAM) of a liquid crystal (LC) selectively on metallic areas in a mixed substrate which comprises both metallic and nonmetallic areas, the process comprising the steps:
ib) spin coating on a mixed substrate any one of the compositions as described wherein said liquid crystal has a polar anchor group (800) selected from the group consisting of a phosphonate ester comprising moiety, a phosphonic acid comprising moiety, a thiol comprising moiety, and an amino comprising moiety and where further said nonmetallic areas are selected from silicon dioxide, silicon with native oxide, silicon nitride, and silicon oxynitride, and said metallic areas are selected from the group consisting of tungsten, gold, silver, copper, cobalt, ruthenium, zirconium, titanium, and hafnium.
iib) baking at a temperature ranging from about 150° C. to about 180° C. for about 2 min to about 10 min under an inert gas,
iiib) rinsing with an organic spin coating solvent,
ivb) air drying substrate,
vb) repeat steps ib) to ivb) two times,
vib) air drying substrate, to obtain a self-assembled monolayer of liquid crystal only on the metallic areas of said substrate.

In another aspect of this process said polar group (800) is selected from ones having ones having structure (IIIp), structure (IVp), structure (Vp), structure (VIp), structure (VIIp), structure (VIIIp) and structure (IXp), as described herein.

In another aspect of this process said liquid crystal is selected from the group consisting of ones having structures (M8), (M9), (M10), (M11), (M12), (M13) and (M14), as described herein. In a more specific embodiment said liquid crystal has structure (M1), in another it has structure (M8), in another it has structure (M9), in another it has structure (M10), in another it has structure (M11), in another it has structure (M12), in another it has structure (M13), and in another it has structure (M14).

Another aspect of this invention is a process for selective atomic layer deposition on metallic areas of a mixed substrate comprising both metallic and non-metallic areas, the process comprising the steps:
ic) producing a mixed substrate in which metallic areas have a self-assembled monolayer of liquid crystal according to the above-described process which employs any one of compositions as described herein where said liquid crystal has a polar anchor group (800) selected from ones having structure (IIIp), structure (IVp), structure (Vp), as described herein, (or any one of compositions which contain the more specific liquid crystals of this type as discussed above),
iic) using an atomic layer deposition technique, deposit a metal oxide more selectively on the non-metallic areas which do not have a self-assembled monolayer of liquid crystal.
   In a more specific aspect of this embodiment, said atomic layer deposition technique is one employing deposition of about 10 to about 50 cycles of deposition, wherein each cycle employs a vapor treatment with (MeCp)₂Hf(OMe)Me for about 1 to about 5 s followed by a treatment with N₂ for about 5 s to about 15 s, a treatment with H₂O for about 1 s to about 5 s, and a treatment with N₂ for about 5 s to about 15 s at a temperature of about 250° C. to about 350° C.

### EXAMPLES

Reference will now be made to more specific embodiments of the present disclosure and experimental results that provide support for such embodiments. The examples are given below to more fully illustrate the disclosed subject matter and should not be construed as limiting the disclosed subject matter in any way.

It will be apparent to those skilled in the art that various modifications and variations can be made in the disclosed subject matter and specific examples provided herein without departing from the spirit or scope of the disclosed subject matter. Thus, it is intended that the disclosed subject matter, including the descriptions provided by the following examples, covers the modifications and variations of the disclosed subject matter that come within the scope of any claims and their equivalents.

Although the disclosed and claimed subject matter has been described and illustrated with a certain degree of particularity, it is understood that the disclosure has been made only by way of example, and that numerous changes in the conditions and order of steps can be resorted to by those skilled in the art without departing from the spirit and scope of the disclosed and claimed subject matter.

### Chemicals

All chemicals unless otherwise indicated were purchased from Sigma Aldrich (3050 Spruce St., St. Louis, MO 63103).

### Instrumentation.

### Ellipsometry

Ellipsometry thickness measurement was done using J. A. Woollam. SAM FTs were measured using a single layer model with RI of organic layer 1.45.

### X-Ray Photoelectron Spectroscopy (XPS) Measurement

These were done using K-alpha from Thermo-Fisher. Experiments were runs using pass energy 50 eVe, step size, 0.100 eV, dwell time 50 ms and 10 scans per element.

### Water Contact Angle (WCA) Measurement

These were done with dynamic contact angle measurement tool from Kruss at room temperature. 4 microliter droplets of deionized water were used. Reported values are average of measurements of 5-6 spots per 1×1 inch coupons.

### DSC Measurements

DSC measurement of glass transition temperature were done using a TA instrument DSC 2500 under nitrogen with a heating and cooling cycles, ramp of 10° C./min. The glass transition temperature (T_{g}) was measured in first heating scan from 0 to 300° C. The midpoint of endothermic transition was considered. In characterizing liquid crystal compounds by DSC the following abbreviations are employed to denote different types physical of liquid crystal transitions: T_{g}=Glass Transition, N=Nematic Phase transition, Sm=Smectic Phase Transition, SmA=Smectic A Phase Transition, SmB=Smectic B Phase Transition, SmC=Smectic C Phase Transition. I=Isotropic Phase Transition.

### ¹H NMR

¹H NMR spectra were recorded using Bruker Advanced III 400 MHz spectrometer in CDCl₃.

### Si/SiOx Wafer

Six- and eight-inch wafers were obtained from Silicon Valley Microelectronics (SVM).

### X-Ray Reflectivity (XRR) Measurements.

These measurements were done with at Darmstadt Technical University, Germany.

Spin-coat monolayer (SML) formulations were prepared by dissolving LCC precursors in EBR7030, a mixture of PGMEA and PGME. 1 wt. % solutions were prepared and filtered using 0.2-micron filter. SAM was prepared on substrates SiOx, copper, tungsten, cobalt, and ruthenium using three step spin-coat-bake-rinse protocol as follows: Solutions were spin-coated at 1500 rpm and baked under nitrogen at 170° C. for 5 min, rinsed with excess EBR, blow air dried and used for further analysis. SML with hydroxyl anchor groups were used to prepare SAM on SiOx. Theoretical and experimental film thickness (ellipsometry, VASE) and WCA for series of SAM are tabulated in table 1. As seen from the table film thickness values are higher than theory values, whereas WCA values are above 90° indicating a hydrophobic surface due to non-polar alkyl tail groups. Film thickness and molecular density was calculated using X-ray reflectometry analysis. As shown in table 2, XRR FT values are lower than ellipsometry. Furthermore, value of molecules per nm² indicates moderate packing density. As a result of moderate packing density this SAM molecules show relatively moderate thermal stability and TGA shows SAM's starts degrading above 270° C. Passivation properties were measured against ALD of hafnium oxide using MeCp)₂Hf(OMe)Me (2 s), N₂ (10 s), H₂O (2 s), N₂ (10 s)/cycle at 300° C. SML200-series SAM's shows passivation up to 50 cycles against ALD of HfOx. Moderate passivation is obtained due to moderate SAM packing density. Comparison of single vs dipodal hydroxyl groups showed that, in order to obtain decent SAM packing density, dipodal hydroxyl groups are needed.

Formulations SML-301, SML-302, SML-402 to SML-407, SML351-SML-352 (using respectively LCC of structures (M8), (M9), (M12), (M17), (M10), (M11)) with thiol, amino, and diethyl phosphonate anchor groups were used for selective SAM deposition on metal surface. SAM's were prepared at bake temperature 170° C. Table 1-3 and figure-5 show selective assembly of SML-302, SML-352, and SML-404 (respectively structure (M9), (M11), (M14) on metal vs SiOx. FIG. 6 show ALD passivation of SML-302 and SML-404 (respectively structures (M9), (M14)) on copper, tungsten, and SiOx. Passivation data indicates SAM packing density follow reactivity order of thiol>amino>diethyl phosphonate. Copper show better packing density than tungsten. SAM packing density reflects passivation properties against ALD of HfOx. Owing to moderate thermal stability of SAM's passivation properties were measured at 200° C. Passivation of SML-302, SML-404 and SML-406 (respectively made from LLC of structure (M9), (M14), and (M16)) copper corresponds ˜6 nm of HfOx. Passivation properties of SML-406 (M16) was compared on different metals which shows Co>Cu>W>Ru, indicating corresponding SAM packing density. FT measured and reported in Tables 1-3 were measured using ellipsometry (VASE). TGA analysis reported herein were done using heat rate 10° C./min. Also measured were water contact angel (WCA).

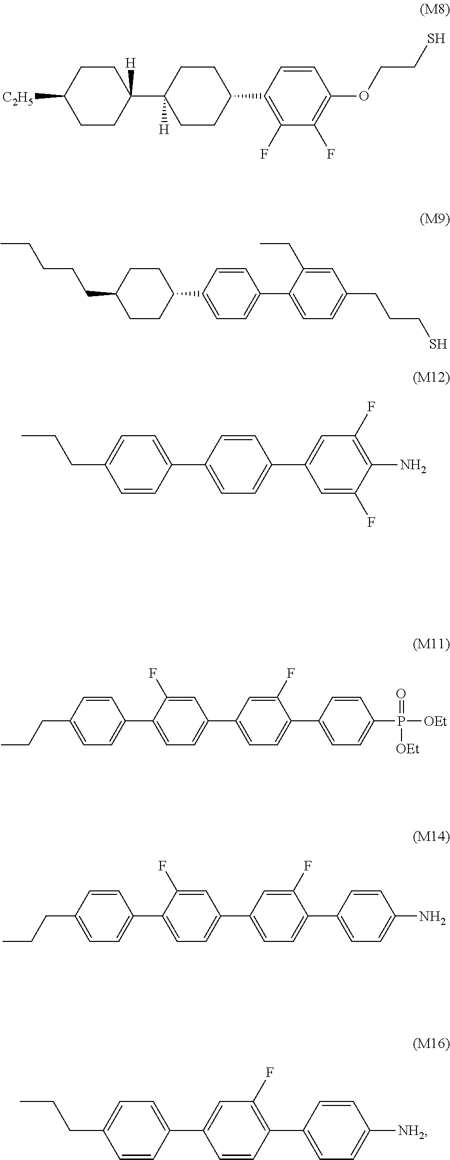 [SMILES: CC[C@H]1CC[C@H]([C@H]2CC[C@H](c3ccc(OCCS)c(F)c3F)CC2)CC1 ; CCCCC[C@H]1CC[C@H](c2ccc(-c3ccc(CCCS)cc3CC)cc2)CC1 ; CCCc1ccc(-c2ccc(-c3cc(F)c(N)c(F)c3)cc2)cc1 ; CCCc1ccc(-c2ccc(-c3ccc(-c4ccc(P(=O)(OCC)OCC)cc4)c(F)c3)cc2F)cc1 ; CCCc1ccc(-c2ccc(-c3ccc(-c4ccc(N)cc4)c(F)c3)cc2F)cc1 ; CCCc1ccc(-c2ccc(-c3ccc(N)cc3)c(F)c2)cc1]

TABLE 1
LCC formulations and SAM properties on SiOx

| LCC | | | FT_{Th}, | FT_{Obs}, | WCA |
|---|---|---|---|---|---|
| Structure | Formulation | Head group | nm | nm | (θ) |
| (M1) | SML-201 | hydroxyl | 2.30 | 2.20 | 92 |
| (M2) | SML-202 | hydroxyl | 1.45 | 1.70 | 96 |
| (M3) | SML-203 | hydroxyl | 2.25 | 2.30 | 96 |
| (M4) | SML-204 | hydroxyl | 1.60 | 1.60 | 97 |
| (M5) | SML-205 | hydroxyl | 2.25 | 2.60 | 110 |
| (M7) | SML-207 | hydroxyl | 2.10 | 1.90 | 102 |
| (M8) | SML-301 | thiol | 1.85 | n/a | n/a |
| (M9) | SML-302 | thiol | 2.20 | n/a | n/a |
| (M10) | SML-351 | diethyl | 2.15 | n/a | n/a |
| | | phosphonate | | | |
| (M11) | SML-352 | diethyl | 2.15 | n/a | n/a |
| | | phosphonate | | | |
| (M12) | SML-402 | amino | 1.65 | n/a | n/a |
| (M13) | SML-403 | amino | 1.65 | n/a | n/a |
| (M14) | SML-404 | amino | 2.05 | n/a | n/a |
| (M15) | SML-405 | amino | 1.70 | n/a | n/a |
| (M16) | SML-406 | amino | 1.65 | n/a | n/a |
| (M15) | SML-407 | amino | 1.85 | n/a | n/a |

FT measured and reported in Table 1 were measured using ellipsometry (VASE). TGA analysis using heat rate 10° C./min, water contact angel (WCA)

TABLE 2
Tabulated values from XRR analysis

| | | | | | | Mole- |
|---|---|---|---|---|---|---|
| | | VASE | XRR, | | Area per | cules |
| For- | FT_{(Theory)} | FT | FT | Electron | molecule | per |
| mulation | nm | (nm) | (nm) | density | (\acute{Å}²) | (nm²) |
| SML-201 | 2.30 | 2.20 | 1.38 | 0.342 | 55.59 | 1.83 |
| SML-202 | 1.45 | 1.70 | 1.23 | 0.363 | 38.16 | 2.72 |
| SML-203 | 2.25 | 2.30 | 1.86 | 0.347 | 37.12 | 2.70 |
| SML-204 | 1.60 | 1.60 | 1.07 | 0.348 | 49.66 | 2.01 |
| SML-205 | 2.25 | 2.60 | 1.67 | 0.372 | 35.94 | 2.78 |
| SML-207 | 2.10 | 1.90 | 1.63 | 0.342 | 37.12 | 2.70 |

FIG. 4 shows the relative stability of SAM's measured via their change in water contact angle (WCA) as a function of increasing temperature. These materials were heated for 5 minutes under nitrogen at each different temperature indicated in this graph. According to these measures these have the following order of thermal stability SML-204>SML-207>SML-202>SML-201>SML-205>SML-203. All SAMs showed good stability up to 250° C. The relative LC-SAM stability is goverend by LC-SAM packing order (ring substitution) and type of a anchor groups. For example, both SML-205 and SML-207 has 1,2 vicinal diols, but C2 spacer in SML-207 show better stability than SML-205 with C3 spacer. SML-204 has two single alcohol groups, which forms two chemical bonds via two separate spacer group has better thermal than SML-207. SML-203 and SML-207 both has vicinal diols and C3 spacer, but SML-203 has ethyl and fluro group on aromatic ring, thereby reducing LC-packing density due to steric hinderance.

FIG. 5 shows the relative stability of SAM containing a polar anchoring group hydroxy moieties as measured via their change in XPS carbon atomic percentage after these SAM's were heated for 5 min under nitrogen at different temperature. XPS analysis show relative C atomic % (at. %) percentage values. SML-205 and SML-207 show higher C at. % due to its molecular composition has higher carbon content. At 250° C. relative change in C at. % is very small for all the SAMs, since after breakage of surface chemical bonds, this SAMs are still present on the surface and contribute to total C at. %.

FIG. 6 shows the results of a study which was done on the Passivation properties of dielectric SAM's against atomic layer deposition of hafnium oxide at 300° C. on SML-201 to SML-207. From this study the most resistant to hafnium oxide deposition after 100 cycles was SML-205 (M5), followed by SML-204 (M4), SML-207 (M7), SML-202 (M2) and SML-201 (M1).

Table 3 shows a water contact study which was done to measure the extent of SAM formation the metal substrates W and Cu compared to Si with native oxide. In this study three SAM materials were compared SML-302 (M9), SML-352 (M11) and SML-404 (M14), which respectively had an thiol, diethyl phosphonate, or a amino anchoring group. From this it was seen that this functional groups selectively reacts to metal surface vs SiOx.

FIG. 7 shows the results of a XPS study which was done on Si, W and Cu substrates. which showed that normalized C at. % is higher for both W and Cu, and very low for Si. Demonstrating this SAMs selectively grafts to metal surface with an order of Cu>>W>>>>Si

TABLE 3
Selective examples SAM deposition on metal surface

| LCC | | SAM WCA (θ) | | |
|---|---|---|---|---|
| Structure | Entry | W | Cu | Si |
| (M9) | SML-302 | 81 | 92 | 24 |
| (M11) | SML-352 | 60 | 79 | 35 |
| (M14) | SML-404 | 81 | 98 | 21 |

FIG. 8 shows the result of a passivation which was done showing the relative passivation properties of SML-302 and SML-404 against ALD of HfOx at 200° C. atter 100, 200 and 300 cycles. This study shows SML 404 show better passivation versus SML-302. Further the order of passivation is Cu>>W>>>>Si.

Table 4 shows the result of water contact experiments which were done to ascertain the selectivity of SML-406 (M16) which had an amino anchoring point on different metal substrates. The results show that relative order of WCA is Ru>W>Cu>>>Si.

FIG. 9 shows the passivation of LC-SAM, SML-406 against atomic layer deposition of HFOx at 200° C. on various metal W, Cu, Co, Ru. These illustrates the relative order of passivation is Co>Cu>>W>>>Ru>>>>Si.

TABLE 4
Selective deposition of SML-406 M16

| | WCA (θ) | | | | |
|---|---|---|---|---|---|
| | W | Cu | Co | Ru | Si |
| SML-406 | 86 | 82 | 63 | 94 | 38 |

### Synthesis of LCC SAM Precursors

### Preparation of LCC of Structure (M1) (SML-201)

### Step 3 in Synthesis of LLC of Structure (M1)

Scheme 1 shows an outline of Step 3 in the synthesis of LCC of structure (M1). Specifically, a solution of 2-[2-[3-ethyl-4-[4-(4-pentylcyclohexyl) phenyl] phenyl] ethyl]-propanedioate (17.86 g, 34.29 mmol) in THF (75 mL) was added dropwise to LiAlH₄ (1.90 g, 50.06 mmol) in toluene (15 mL). The mixture was stirred for 1 h at 65° C. before a small amount of water, methyl tert-butyl ether (MTBE), and diluted HCl were added. The phases were separated, and the aqueous phase was extracted with MTBE. The combined organic phases were dried over Na₂SO₄, filtered off, and the solvent was removed under reduced pressure. Further purification by column chromatography (silica gel, heptane/ethyl acetate: 1/4) gave diethyl 2-[2-[3-ethyl-4-[4-(4-pentylcyclohexyl)phenyl] phenyl] ethyl]-propanedioate as a colorless solid (11.0 g, 25.19 mmol, HPLC: 99.4%) in 73% yield. ¹H-NMR (CDCl₃, 500 MHz): δ=7.23-7.19 (m, 4H), 7.13-7.11 (m, 2H), 7.05-7.03 (m, 1H), 3.91-3.87 (m, 2H), 3.77-3.72 (m, 2H), 2.73-2.65 (m, 2H), 2.58 (q, J=7.5 Hz, 2H), 2.53-2.47 (m, 1H), 2.16-2.11 (m, 2H), 1.99-1.92 (m, 2H), 1.89-1.82 (m, 3H), 1.71-1.62 (m, 2H), 1.49 (qd, J=12.8 Hz, J=3.3 Hz, 2H), 1.36-1.20 (m, 9H), 1.10 (t, J=7.5 Hz, 3H), 1.09-1.07 (m, 1H), 0.90 (t, J=7.0 Hz, 3H). APCI-MS: m/z: 437.3. DSC: T_{g}−67° C., 79° C. Sm, 112° C. SmA, 122° C. I. TGA: T_{5% loss}=303° C.

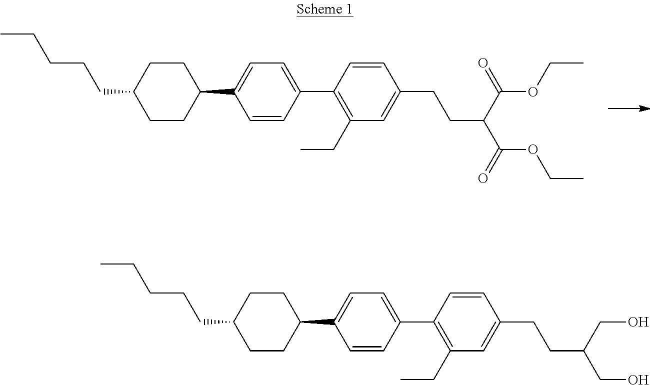

### Preparation of LCC of Structure (M1) (SML-201)

### Step 3 in Synthesis of LCC of Structure (M1)

Scheme 2 shows a general outline of the second step in the synthesis of LCC of structure (M1), specifically, diethyl malonate (6.4 mL, 0.042 mol) and 2-[3-ethyl-4-[4-(4-pentylcyclohexyl)phenyl] phenyl]ethyl methanesulfonate (9.6 g, 0.021 mol) was added to a solution of sodium ethylate (2.8 g, 0.042 mol) in ethanol (30 mL) and stirred over night at 75° C. The mixture allowed to cool down to room temperature before water was added. The aqueous phase was extracted with MTBE and the combined organic phases were dried over Na₂SO₄, filtered off, and the solvent was removed under reduced pressure. Further purification by column chromatography (silica gel, heptane/ethyl acetate: 9/1) gave diethyl 2-[2-[3-ethyl-4-[4-(4-pentylcyclohexyl)phenyl]-phenyl]ethyl]propanedioate as a colorless oil (11.8 g, 0.011 mol, HPLC: 99.0%) in 53% yield.

¹H-NMR (CDCl₃, 500 MHz): δ=7.23-7.19 (m, 4H), 7.13-7.11 (m, 2H), 7.05-7.03 (m, 1H), 4.24-4.19 (m, 4H), 3.39 (t, J=7.5 Hz, 2H), 2.68 (dd, J=8.9 Hz, J=6.7 Hz, 2H), 2.58 (q, J=7.5 Hz, 2H), 2.53-2.47 (m, 1H), 2.28-2.24 (m, 2H), 1.95-1.87 (m, 4H), 1.52-1.47 (m, 2H), 1.34-1.22 (m, 17H), 1.10 (t, J=7.5 Hz, 3H), 1.09-1.07 (m, 1H), 0.90 (t, J=7.5 Hz, 3H). EI-MS: m/z: 520.4.

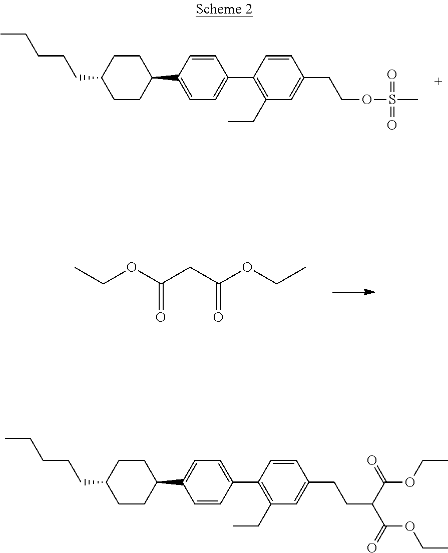

### Step 2 in Synthesis of LCC of Structure (M1)

Scheme 3 shows a general outline of the second step in the synthesis of LCC of structure (M1), specifically, pyridine (15.9 mL, 197.0 mmol) was added to a solution of 2-[2-Ethyl-4′-(4-pentyl-cyclohexyl)-biphenyl-4-yl]-ethanol (13.7 g, 35.83 mmol) and 4-(dimethylamino)-pyridine (0.9 g, 7.17 mmol) in dichloromethane (70 mL). Methanesulfonyl chloride (8.3 mL, 107.5 mmol) was added at 0° C. and the resulting solution was stirred overnight at room temperature. The mixture was diluted with water and the aqueous phase was extracted with dichloro-methane and the combined organic phases were washed with 2N HCl and water and dried over sodium sulphate. Further purification by column chromatography (silica gel, dichloromethane) gave 2-[3-ethyl-4-[4-(4-pentylcyclohexyl)phenyl] phenyl]ethyl methanesulfonate as a colorless solid (13.3 g, 29.0 mmol, HPLC: 99.3%) in 81% yield.

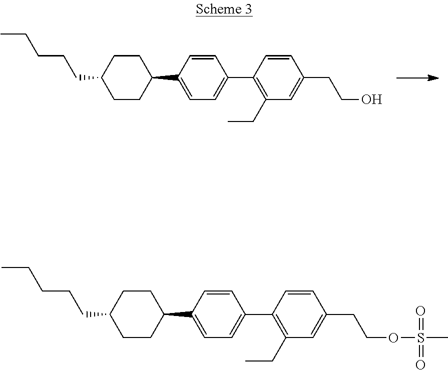 [SMILES: CCCCC[C@H]1CC[C@H](c2ccc(-c3ccc(CCO)cc3CC)cc2)CC1 ; CCCCC[C@H]1CC[C@H](c2ccc(-c3ccc(CCOS(C)(=O)=O)cc3CC)cc2)CC1]

### Step 1 in Synthesis of LCC of Structure (M1)

Scheme 4 shows a general outline of the first step in the synthesis of LCC of structure (M1), specifically, a very small amount of iodine was added to a mixture of Mg (1.97 g, 0.081 mol) in THF (10 mL) and heated to 50° C. A solution of 4-bromo-2-ethyl-4′-(4-pentyl-cyclohexyl)-biphenyl (24.6 g, 0.058 mol) (prepared as described in Yano, Kyoichi et al., JP 2017025007) in THF (180 mL) was added and the mixture was stirred for 3 h at 70° C. After cooling down to −20° C., a cold solution of ethylene oxide (3.32 g, 0.081 mol) in THF (20 mL) was slowly added and the mixture was stirred for another 30 min at −20° C. The mixture was diluted with THF (200 mL) and stirred over night at room temperature. After cooling down to −5° C., water (600 mL) was slowly added. The resulting precipitate was dissolved by adding HCl. MTBE was added and the organic phase was separated. The aqueous phase was extracted with MTBE and the combined organic phases were dried over Na₂SO₄, filtered off, and the solvent was removed under reduced pressure. Further purification by column chromatography (silica gel; heptane/ethyl acetate: 9/1) yielded 2-[2-Ethyl-4′-(4-pentyl-cyclohexyl)-biphenyl-4-yl]-ethanol (21.8 g, 0.057 mol. 99% yield, HPLC: 99.0%

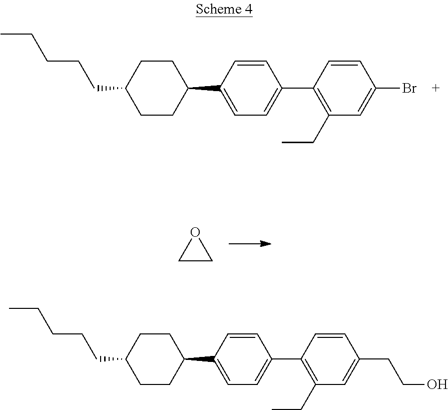

### Preparation of LCC of Structure (M2) (SML-202)

### Step 4 in Synthesis of LCC of Structure (M2)

Scheme 5 shows a general outline of the fourth step in the synthesis of LCC of structure (M2), specifically, a solution of 2-[2-(2-fluoro-4′-propyl-biphenyl-4-yl)-ethyl]-malonic acid diethyl ester (4.80 g, 11.20 mmol) in THF (20 mL) was added dropwise to LiAlH₄ (0.55 g, 14.49 mmol) in toluene (4 mL). The mixture was stirred for 1 h at 65° C. before a small amount of water, MTBE, and diluted HCL were added. The phases were separated, and the aqueous phase was extracted with MTBE. The combined organic phases were dried over Na₂SO₄, filtered off, and the solvent was removed under reduced pressure. Recrystallization from heptane provided pure 2-[2-(2-fluoro-4′-propyl-biphenyl-4-yl)-ethyl]-propane-1,3-diol as a colorless solid (1.70 g, 5.37 mmol, HPLC: 99.6%) in 45% yield. ¹H-NMR (DMSO-d₆, 400 MHz): δ=7.46-7.37 (m, 3H), 7.28 (d, J=8.1 Hz, 2H), 7.15-7.09 (m, 2H), 4.43 (t, J=5.2 Hz, 2H), 2.66 (dd, J=9.2 Hz, J=6.6 Hz, 2H), 2.59 (t, J=7.6 Hz, 2H), 1.69-1.47 (m, 5H), 0.92 (t, J=7.3 Hz, 3H). EI-MS: m/z: 316.1. DSC: 66° C. SmC, 73° C. I. TGA: T_{5% loss}=258° C.

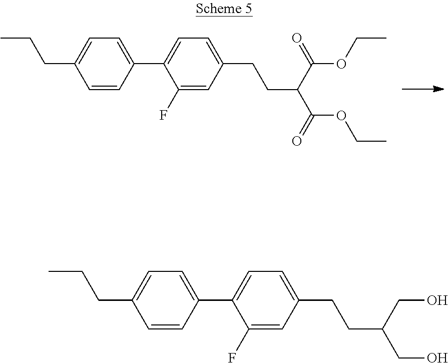

### Preparation of LCC of Structure (M2) (SML-202)

### Step 3 in Synthesis of LLC of Structure (M2)

Scheme 6 shows a general outline of the third step in the synthesis of LCC of structure (M2), specifically, diethyl malonate (6.4 mL, 0.06 mol) and sodium ethylate (24.00 mL, 0.06 mol, 20% solution in ethanol) were added to a stirred solution of 4-(2-Bromo-ethyl)-2-fluoro-4′-propyl-biphenyl (10.00 g, 0.03 mol) in ethanol (11 mL). After stirring over night at 75° C., the mixture allowed to cool down to room temperature and water was added. The aqueous phase was extracted with MTBE and the combined organic phases were dried over Na₂SO₄, filtered off, and the solvent was removed under reduced pressure. Further purification by column chromatography (silica gel, heptane/ethyl acetate: 9/1) gave 2-[2-(2-fluoro-4′-propyl-biphenyl-4-yl)-ethyl]-malonic acid diethyl ester as a colorless oil (4.8 g, 0.01 mol, GC: 99.7%) in 40% yield.

¹H-NMR (CDCl₃, 500 MHz): δ=7.45 (dq, J=8.4 Hz, J=2.1 Hz, 2H), 7.38 (t, J=8.0 Hz, 1H), 7.24 (d, J=8.2 Hz, 2H), 7.02 (dd, J=7.8 Hz, J=1.7 Hz, 1H), 6.98 (dd, J=11.6 Hz, J=1.7 Hz, 1H), 4.24-4.15 (m, 7H), 2.69 (dd, J=8.8 Hz, J=6.5 Hz, 2H), 2.62 (dd, J=8.6 Hz, J=6.8 Hz, 2H), 1.72-1.62 (m, 2H), 1.28 (t, J=7.2 Hz, 6H), 0.97 (t, J=7.3 Hz, 3H). EI-MS: m/z: 400.2.

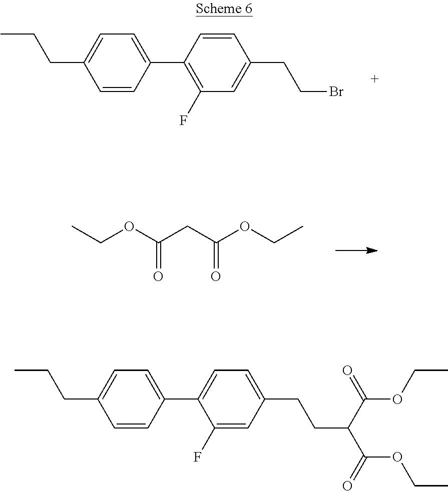 [SMILES: CCCc1ccc(-c2ccc(CCBr)cc2F)cc1 ; CCOC(=O)CC(=O)OCC ; CCCc1ccc(-c2ccc(CCC(C(=O)OCC)C(=O)OCC)cc2F)cc1]

### Step 2 in Synthesis of LCC of Structure (M2)

Scheme 7 shows a general outline of the second step in the synthesis of LCC of structure (M2), specifically, 2-(2-Fluoro-4′-propyl-biphenyl-4-yl)-ethanol (11.6 g, 0.04 mol) was refluxed in HBr (40.00 mL, 0.35 mol, 47%) for 16 h. Water and MTBE were added to the cooled reaction mixture and the organic phase was washed with water and NaHCO₃ solution. Further purification by column chromatography (silica gel, heptane) gave 4-(2-Bromo-ethyl)-2-fluoro-4′-propyl-biphenyl (10.0 g, 0.03 mmol) in 69% yield.

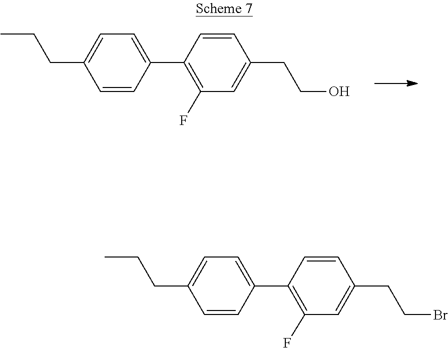 [SMILES: CCCc1ccc(-c2ccc(CCO)cc2F)cc1 ; CCCc1ccc(-c2ccc(CCBr)cc2F)cc1]

### Step 1 in Synthesis of LCC of Structure (M2)

Scheme 8 shows a general outline of the first step in the synthesis of LCC of structure (M2), specifically, n-BuLi (88.00 mL, 0.14 mol, 15% in hexane) was added dropwise to a solution of 4-bromo-2-fluoro-4′-propyl-1,1′-biphenyl (41.10 g, 0.14 mol) in diethyl ether (400 mL) at −78° C. and stirred for another 30 min at this temperature. Ethylene oxide (5.17 mL, 0.11 mol) was added at −78° C. After stirring 1 h at −78° C., BF₃·Et₂O (27.90 mL, 0.11 mol, 48%) was added within 1 h. The solution was now allowed to warm up to −20° C., quenched with saturated ammonium chloride solution, and the aqueous phase was extracted with MTBE. The combined organic phases were dried over Na₂SO₄, filtered off, and the solvent was removed under reduced pressure. 2-(2-Fluoro-4′-propyl-biphenyl-4-yl)-ethanol (12.6 g, 0.05 mol) was obtained as a colourless solid in 46% yield after purification by column chromatography. ¹H-NMR (CDCl₃, 500 MHz): δ=7.46 (dq, J=8.4 Hz, J=2.1 Hz, 2H), 7.38 (t, J=8.0 Hz, 1H), 7.26 (d, J=8.2 Hz, 2H), 7.07 (dd, J=7.8 Hz, J=1.8 Hz, 1H), 7.04 (dd, J=11.5 Hz, J=1.7 Hz, 1H), 3.91 (q, J=6.4 Hz, 2H), 2.90 (t, J=6.5 Hz, 2H), 2.63 (dd, J=8.6 Hz, J=6.8 Hz, 2H), 1.76-1.62 (m, 2H), 1.51 (t, J=5.8 Hz, 1H), 0.98 (t, J=7.3 Hz, 3H). EI-MS: m/z: 258.0

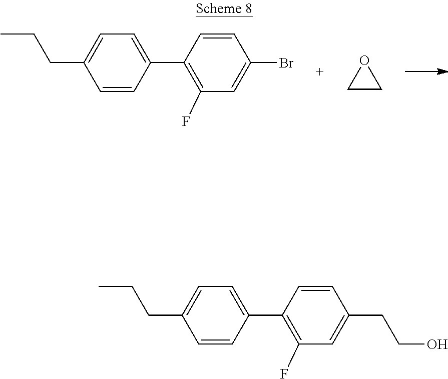

### Preparation of LCC of Structure of (M3) (SML-203)

The LCC of structure (M3), 2-[2-[3-ethyl-4-[2-fluoro-4-[2-(4-pentylphenyl)ethyl] phenyl]phenyl]ethyl]-propane-1,3-diol was prepared as described in Archetti, Graziano et al., WO 2014/094959 A1.

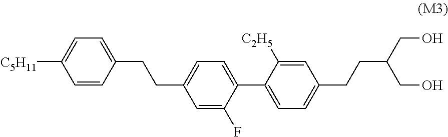

Preparation of LCC of structure (M4) (SML-204)

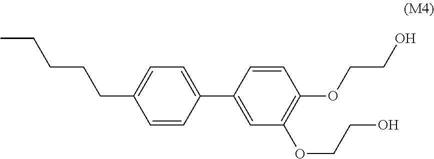 [SMILES: CCCCCc1ccc(-c2ccc(OCCO)c(OCCO)c2)cc1]

### Step 3 in Synthesis of LCC of Structure (M4)

In the third step in the synthesis of (M4), tetra-n-butyl ammonium fluoride (35.00 mL, 35.00 mmol, 1M THF solution) was added to a solution of tert-butyl-[2-[2-[2-[tert-butyl-(dimethyl) silyl]oxy-ethoxy]-4-(4-pentylphenyl) phenoxy]ethoxy]-dimethyl-silane (8.50 g, 14.69 mmol) in THF (100 mL). The solution was stirred for 2 h at room temperature before it was poured into water, acidified with 2N HCl, and extracted with ethyl acetate. Further purification by recrystallization from toluene/ethyl acetate and column chromatography (silica gel, toluene/ethyl acetate: 1/3) gave 2-[2-(2-hydroxyethoxy)-4-(4-pentylphenyl) phenoxy]ethanol as a colorless solid (3.30 g, 9.55 mmol, HPLC: 99.7%) in 65% yield.

¹H-NMR (CDCl₃, 500 MHz): δ=7.45 (d, J=8.2 Hz, 2H), 7.22 (d, J=8.2 Hz, 2H), 7.21-7.12 (m, 2H), 6.99 (d, J=8.3 Hz, 1H), 4.43 (td, J=6.1 Hz, J=2.0 Hz, 2H), 4.16-4.12 (m, 4H), 3.92-3.89 (m, 4H), 2.62 (t, J=7.7 Hz, 2H), 1.69-1.60 (m, 2H), 1.41-1.28 (m, 4H), 0.90 (t, J=7.0 Hz, 3H). EI-MS: m/z: 344.1. DSC: 128° C. I. TGA: T_{5% loss}=273° C.

### Step 2 in Synthesis of LCC of Structure (M4)

Scheme 9 shows a general outline of the second step in the synthesis of LCC of structure (M4), specifically, 2-[4-Bromo-2-[2-[tert-butyl(dimethyl) silyl]oxyethoxy]-phenoxy]ethoxy-tert-butyl-dimethyl-silane (13.5 g, 25.36 mmol) and (4-Pentylphenyl) boronic acid (5.00 g, 26.03 mmol) were added to a stirred solution of BNaO₂·4H₂O (5.73 g, 0.04 mol) in water (40 mL). Pd(PPh₃)₂Cl₂ (1.04 g, 1.45 mmol) and THF (150 mL) and the mixture was stirred at 70° C. over night. The mixture allowed to cool down to room temperature before water was added. The aqueous phase was extracted with ethyl acetate and the combined organic phases were dried over Na₂SO₄, filtered off, and the solvent was removed under reduced pressure. Further purification by column chromatography (silica gel, heptane/ethyl acetate: 95/5) gave tert-butyl-[2-[2-[2-[tert-butyl-(dimethyl) silyl]oxyethoxy]-4-(4-pentylphenyl) phenoxy]ethoxy]-dimethyl-silane as a colorless oil (8.70 g, 15.03 mmol, HPLC: 99.0%) in 58% yield. EI-MS: m/z: 572.4.

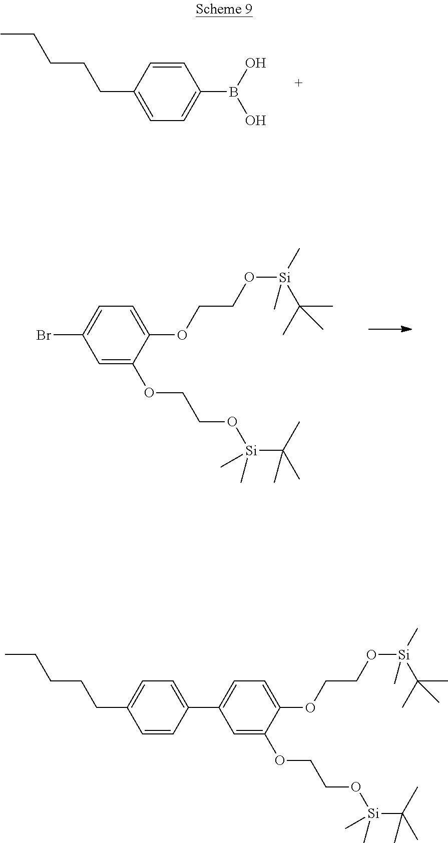

### Step 2 in Synthesis of LCC of Structure (M4)

Scheme 10 shows a general outline of the first step in the synthesis of LCC of structure (M4), specifically, a solution of 4-bromobenzene-1,2-diol (10.00 g, 52.91 mmol) in DMF (50 mL) was added dropwise to NaH (5.00 g, 208.35 mmol) in DMF (170 mL) at 0° C. The reaction mixture was stirred for 2 h at room temperature before (2-bromoethoxy) (tert-butyl)dimethylsilane dissolved in DMF (30 mL) was added. After stirring for 3 d at room temperature, the reaction mixture was poured into water, acidified with 2N HCl, and extracted with MTBE. The combined organic phases were washed with water and dried over sodium sulphate. Further purification by column chromatography (silica gel, heptane/ethyl acetate: 95/5) gave 2-[4-bromo-2-[2-[tert-butyl(dimethyl)silyl]oxyethoxy]-phenoxy]ethoxy-tert-butyl-dimethyl-silane as a colorless oil (18.2 g, 35.27 mmol, GC: 98%) in 67% yield. ¹H-NMR (CDCl₃, 500 MHz): δ=6.95 (d, J=2.3 Hz, 1H), 6.90 (dd, J=8.5, 2.3 Hz, 1H), 6.69 (d, J=8.5 Hz, 1H), 3.99-3.92 (m, 4H), 3.90-3.83 (m, 4H), 0.81 (s, 9H), 0.80 (s, 9H), 0.01 (s, 6H), 0.00 (s, 6H).

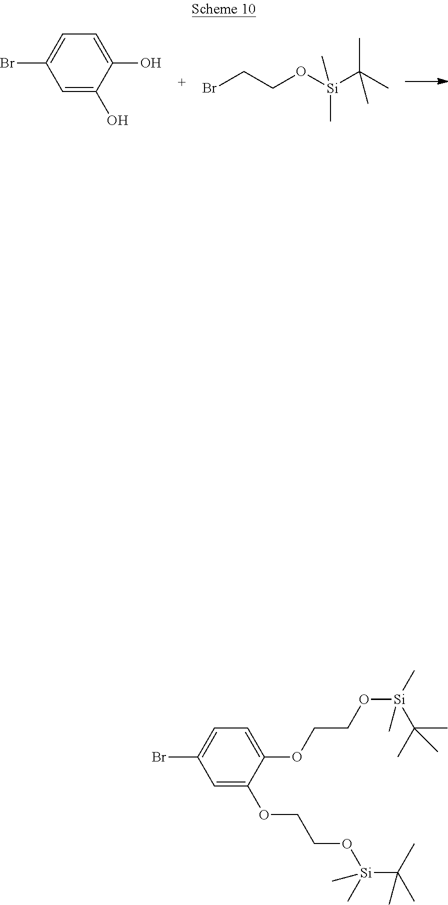 [SMILES: Oc1ccc(Br)cc1O ; CC(C)(C)[Si](C)(C)OCCBr ; CC(C)(C)[Si](C)(C)OCCOc1ccc(Br)cc1OCCO[Si](C)(C)C(C)(C)C]

### Synthesis of LCC of Structure (M5) (SML-205)

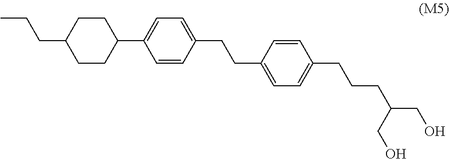 [SMILES: CCCC1CCC(c2ccc(CCc3ccc(CCCC(CO)CO)cc3)cc2)CC1]

The LCC of structure (M5) was prepared using the general procedure employed for the LCC of structure (M3) using appropriate reagents. ¹H-NMR (DMSO-d₆, 500 MHz): δ=7.19-7.04 (m, 8H), 4.27 (t, J=5.1 Hz, 2H), 3.43-3.30 (m, 4H), 2.80 (s, 4H), 2.52-2.50 (m, 5H), 2.41 (tt, J=12.0 Hz, J=3.3 Hz, 1H), 1.84-1.72 (m, 4H), 1.62-1.52 (m, 2H), 1.50-1.14 (m, 11H), 1.03 (qd, J=13.8, 13.3, 3.7 Hz, 2H), 0.89 (t, J=7.3 Hz, 3H). EI-MS: m/z: 422.3. DSC: 155° C. I.

### Synthesis of LCC of Structure (M7) (SML-207)

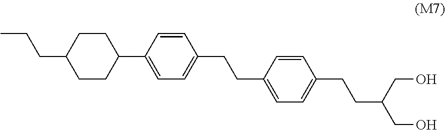 [SMILES: CCCC1CCC(c2ccc(CCc3ccc(CCC(CO)CO)cc3)cc2)CC1]

The LCC of structure (M5) was prepared using the general procedure employed for the LCC of structure (M3) using appropriate reagents. ¹H-NMR (CDCl₃, 500 MHz): δ=7.15-7.07 (m, 8H), 3.87-3.82 (m, 2H), 3.75-3.68 (m, 2H), 2.87 (s, 4H), 2.67-2.60 (m, 2H), 2.44 (tt, J=12.2 Hz, J=3.3 Hz, 1H), 2.14 (t, J=5.1 Hz, 2H), 1.93-1.75 (m, 5H), 1.64-1.58 (m, 2H), 1.49-1.17 (m, 7H), 1.10-0.98 (m, 2H), 0.90 (t, J=7.3 Hz, 3H). EI-MS: m/z: 408.3. DSC: 163° C. SmB, 173° C. SmA, 178° C. I.

### Synthesis of LLC of Structure (M8) (SML-301)

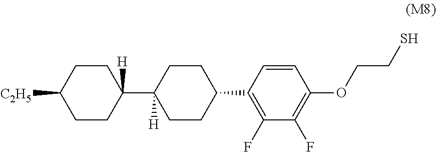 [SMILES: CC[C@H]1CC[C@H]([C@H]2CC[C@H](c3ccc(OCCS)c(F)c3F)CC2)CC1]

The LCC of structure (M8) 2-[4-[4-(4-ethylcyclohexyl) cyclohexyl]-2,3-difluoro-phenoxy]ethanethiol is available was prepared according to the procedure of Yun, Yong-Kuk et al., WO 2017045740.

### Synthesis of LCC of Structure (M9) (SML-302)

### Step 3 in Synthesis of Structure (M9)

Scheme 11 shows a general outline of the third step in the synthesis of LCC of structure (M9), specifically, A suspension of thioacetic acid S-{3-[2-ethyl-4′-(4-pentyl-cyclohexyl)-biphenyl-4-yl]-propyl} ester (12.4 g, 27.5 mmol) in methanol (500 mL) was cooled down to 2° C. Sodium methylate (30% in methanol, 25 mL, 134.7 mmol) was added and the reaction mixture was stirred for 1 h at 0° C. After stirring for another 1 h at 15° C., the reaction mixture was diluted with acetic acid (50% solution) and n-heptane. The aqueous phase was extracted with n-heptane. The combined organic phases were dried over Na₂SO₄ and filtrated. Further purification by column chromatography (silica gel, chlorobutane/heptane: 1/1 and flash chromatography (reverse phase, methyl-tert-butyl ether) gave 3-[2-ethyl-4′-(4-pentyl-cyclohexyl)-biphenyl-4-yl]-propane-1-thiol as a colourless oil (8.3 g, 20.3 mmol, HPLC: 99.7%) in 74% yield. ¹H-NMR (CDCl₃, 500 MHz): δ=7.26-7.22 (m, 4H), 7.16-7.12 (m, 2H), 7.06 (dd, J=7.7 Hz, J=1.9 Hz, 1H), 2.77 (t, J=7.5 Hz, 2H), 2.62 (q, J=7.5 Hz, 4H), 2.53 (tt, J=12.1 Hz, J=3.4 Hz, 1H), 2.05-1.95 (m, 4H), 1.94-1.90 (m, 2H), 1.57-1.46 (m, 2H), 1.42-1.23 (m, 11H), 1.13 (t, J=7.5 Hz, 3H), 0.93 (t, J=7.0 Hz, 3H). EI-MS: m/z: 408.3. DSC: Tg −64° C., 22° C. N, (−33.8° C.) I. TGA: T_{5% loss}=294° C.

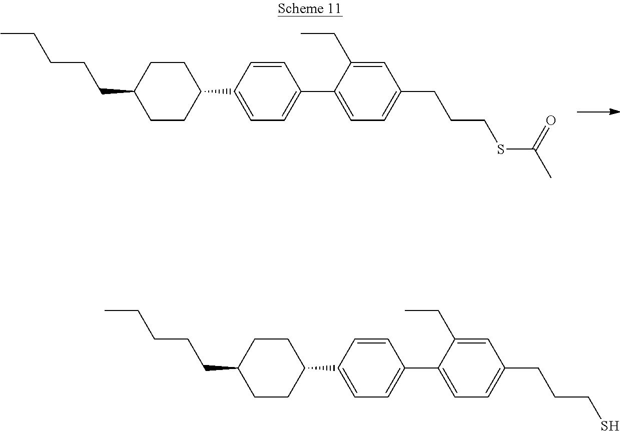

### Step 2 in Synthesis of LCC of Structure (M9)

Scheme 12 shows a general outline of the second step in the synthesis of LCC of structure (M9), A solution of methanesulfonic acid 3-[2-ethyl-4′-(4-pentyl-cyclohexyl)-biphenyl-4-yl]-propyl ester (18.7 g, 39.7 mmol) in DMF (200.0 mL) was added dropwise to a suspension of sodium thioacetate (33.8 g, 262.70 mmol) in DMF (100 mL) and stirred for 0.5 h at room temperature. The reaction mixture was poured into a mixture of toluene and water. The phases were separated, and the aqueous phase was extracted with toluene. The combined organic phases were washed with saturated NaCl solution and dried over sodium sulphate. Further purification by column chromatography (silica gel, toluene) gave thioacetic acid S-{3-[2-ethyl-4′-(4-pentyl-cyclohexyl)-biphenyl-4-yl]-propyl} ester as a brownish oil (12.5 g, 27.1 mmol, HPLC: 97.7%) in 68% yield.

¹H-NMR (CDCl₃, 500 MHz): δ=7.26-7.22 (m, 4H), 7.16-7.12 (m, 2H), 7.06 (dd, J=7.7 Hz, J=1.9 Hz, 1H), 2.96 (t, J=7.3 Hz, 2H), 2.73 (dd, J=8.6 Hz, J=6.8 Hz, 2H), 2.61 (q, J=7.5 Hz, 2H), 2.53 (tt, J=12.2 Hz, J=3.4 Hz, 1H), 2.38 (s, 3H), 2.01-1.84 (m, 6H), 1.56-1.47 (m, 2H), 1.38-1.25 (m, 8H), 1.12 (t, J=7.5 Hz, 3H), 0.93 (t, J=7.0 Hz, 3H).

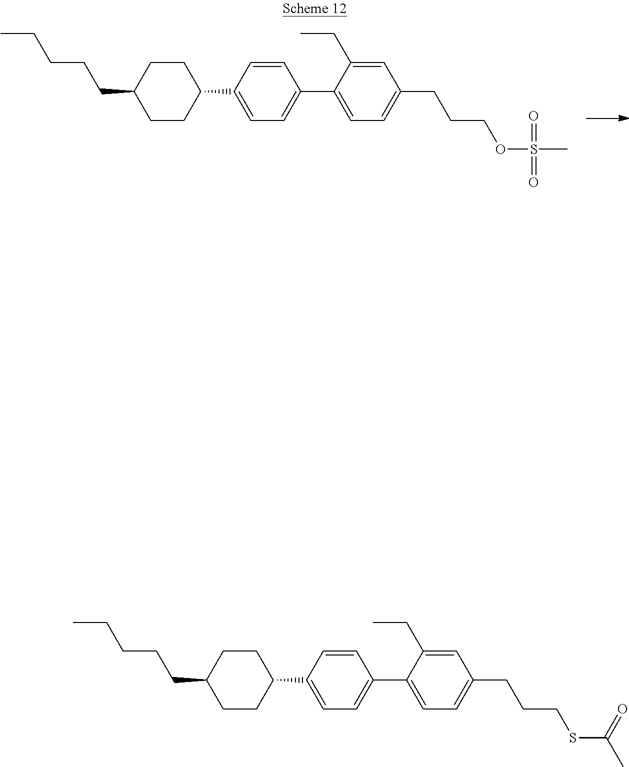

### Step 1 in Synthesis of LCC of Structure (M9)

Scheme 13 shows a general outline of the first step in the synthesis of LCC of structure (M9),

Pyridine (8.6 mL, 105.6 mmol) was added to a solution of 3-[2-ethyl-4′-(4-pentyl-cyclohexyl)-biphenyl-4-yl]-propan-1-ol (20.0 g, 50.94 mmol) [3-[2-Ethyl-4′-(4-pentyl-cyclohexyl)-biphenyl-4-yl]-propan-1-ol is available according to EP2883934] and 4-(dimethylamino)-pyridine (0.6 g, 4.91 mmol) in dichloromethane (200 mL). Methanesulfonyl chloride (4.3 mL, 55.5 mmol) was added at 0° C. and the resulting solution was stirred overnight at room temperature. Pyridine (5.0 mL, 62.0 mmol) and methanesulfonyl chloride (2.0 mL, 25.8 mmol) were added and the mixture was stirred for another 3 days at room temperature. The mixture was poured into diluted hydrochloric acid and stirred for 1 h at room temperature. The aqueous phase was extracted with dichloromethane and the combined organic phases were washed and dried over sodium sulphate. Further purification by column chromatography (silica gel, heptane/ethyl acetate: 8/2) gave methanesulfonic acid 3-[2-ethyl-4′-(4-pentyl-cyclohexyl)-biphenyl-4-yl]-propyl ester as a colorless solid (17.3 g, 36.6 mmol, HPLC: 99.7%) in 72% yield. ¹H-NMR (CDCl₃, 500 MHz): δ=7.26-7.22 (m, 4H), 7.16-7.12 (m, 2H), 7.06 (dd, J=7.7 Hz, J=1.9 Hz, 1H), 4.31 (t, J=6.9 Hz, 2H), 3.04 (s, 1H), 2.80 (t, J=7.5 Hz, 2H), 2.62 (q, J=7.5 Hz, 2H), 2.57-2.48 (m, 1H), 2.18-2.12 (m, 2H), 1.99-1.90 (m, 6H), 1.57-1.44 (m, 4H), 1.38-1.24 (m, 8H), 1.13 (t, J=7.6 Hz, 3H), 0.93 (t, J=7.1 Hz, 3H).

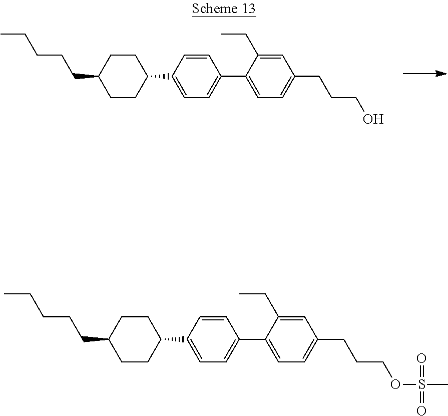 [SMILES: CCCCC[C@H]1CC[C@H](c2ccc(-c3ccc(CCCO)cc3CC)cc2)CC1 ; CCCCC[C@H]1CC[C@H](c2ccc(-c3ccc(CCCOS(C)(=O)=O)cc3CC)cc2)CC1]

### Synthesis of LCC of Structure (M10) (SML-351)

### Step 2 in Synthesis of LCC of Structure (M10)

Scheme 14 shows a general outline of the second step in the synthesis of LCC of structure (M10), specifically, Triethyl phosphite (12.5 g, 0.07 mol) was added to 4-(3-bromopropyl)-2-ethyl-4′-(4-pentylcyclohexyl)-1,1′-biphenyl (11.5 g, 0.02 mol) and stirred for 22 h at 160° C. The excess of triethylphosphite was removed at reduced pressure. Further purification by column chromatography (silica gel; dichloromethane/THF 9:1) gave diethyl[3-[2-ethyl-4′-(4-pentylcyclohexyl)-[1,1′-bisphenyl]-4-yl]propyl]phosphonate (5.5 g, 0.01 mol, GC: 89%) in 38% yield.

¹H-NMR (CDCl₃, 500 MHz): δ=7.26-7.22 (m, 4H), 7.16-7.12 (m, 2H), 7.06-7.04 (m, 1H), 4.11-4.06 (m, 4H), 2.74 (t, J=7.6 Hz, 2H), 2.61 (q, J=7.6 Hz, 2H), 2.53 (m, 1H), 2.09-1.96 (m, 4H), 1.93-1.90 (m, 2H), 1.90-1.82 (m, 4H), 1.57-1.48 (m, 2H), 1.38-1.29 (m, 17H), 1.13 (t, J=7.5 Hz, 3H), 0.93 (t, J=7.0 Hz, 3H).

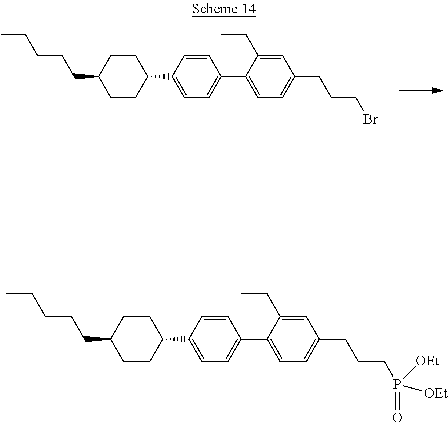 [SMILES: CCCCC[C@H]1CC[C@H](c2ccc(-c3ccc(CCCBr)cc3CC)cc2)CC1 ; CCCCC[C@H]1CC[C@H](c2ccc(-c3ccc(CCCP(=O)(OCC)OCC)cc3CC)cc2)CC1]

### Step 1 in Synthesis of LCC of Structure (M10)

Scheme 15 shows a general outline of the second step in the synthesis of LCC of structure (M10), specifically, triphenylphospine (13.3 g, 11.2 mL, 0.05 mol) was added to a solution of 2-[2-ethyl-4′-(4-pentyl-cyclohexyl)-biphenyl-4-yl]-ethanol (10.0 g, 0.03 mol) and tetrabromomethane (17.0 g, 5.8 mL, 005 mol) in THF (41 mL) at 0° C. The mixture allowed to warm up to room temperature and stirred for 16 h at this temperature. Methyl-tert-butyl ether (50 mL) was added and the phases were separated. The aqueous phase was extracted with methyl-tert-butyl ether and the combined organic phases were dried over Na₂SO₄. Further purification by column chromatography (silica gel, dichloromethane) yielded 4-(3-bromopropyl)-2-ethyl-4′-(4-pentylcyclohexyl)-1,1′-biphenyl (11.5 g, 0.02 mol, GC: 99.3%) in 98% yield.

¹H-NMR (DMSO-d₆, 500 MHz): δ=7.28-7.26 (m, 2H), 7.20-7.16 (m, 3H), 7.08-7.05 (m, 2H), 3.62-3.49 (m, 2H), 2.76-2.70 (m, 2H), 2.56-2.51 (m, 3H), 2.17-2.09 (m, 2H), 1.90-1.80 (m, 4H), 1.52-1.46 (m, 2H), 1.36-1.16 (m, 9H), 1.07-1.02 (m, 5H), 0.88 (t, J=7.0 Hz, 3H).

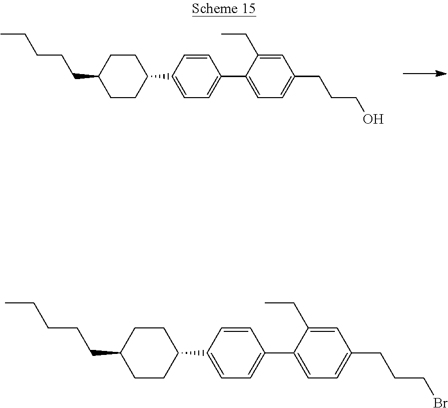 [SMILES: CCCCC[C@H]1CC[C@H](c2ccc(-c3ccc(CCCO)cc3CC)cc2)CC1 ; CCCCC[C@H]1CC[C@H](c2ccc(-c3ccc(CCCBr)cc3CC)cc2)CC1]

### Synthesis of LCC of Structure (M11) (SML-352)

Scheme 16 shows a general outline of the second step in the synthesis of LCC of structure (M11), specifically, 2′-fluoro-4′-{2-fluoro-4′-propyl-[1,1′-biphenyl]-4-yl}-[1,1′-biphenyl]-4-amine (2.80 g, 0.70 mmol) was added to a solution of Et₂O·BF₃ (0.55 mmol, 56 μL) in 48 mL of anhydrous THF at −15° C. Tert-butyl nitrite (3.70 mL, 90%, 3.11 mmol) was added dropwise and the mixture was allowed to slowly warm up to 5° C. The reaction mixture was stirred at room temperature and stirred until all starting aniline consumed. After removal of the solvent under reduced pressure, anhydrous acetonitrile (146 mL), KI (11.62 g, 0.07 mol), Pd(OAc)₂ (167 mg, 0.12 mmol), P(OEt)₃ (5.82 g, 3.50 mmol), and Cs₂CO₃ (15,51 g, 0.05 mol) were added and the reaction mixture was stirred for 4 h at 80° C. under the absence of light. Further purification by column chromatography (silica gel, dichloromethane/THF: 9/1) and recrystallization from toluene provided 4-[4-(4-diethoxyphosphorylphenyl)-3-fluoro-phenyl]-2-fluoro-1-(4-propylphenyl)benzene (1.04 g, 0.20 mmol, GC: 96%) as a light yellow solid in 28% yield. ¹H-NMR (CDCl₃, 500 MHz): δ=7.96-7.92 (m, 2H), 7.75-7.73 (m, 2H), 7.59-7.43 (m, 8H), 7.32 (d, J=8.2 Hz, 2H), 4.26-4.10 (m, 4H), 2.69-2.66 (m, 2H), 1.76-1.69 (m, 2H), 1.39 (t, J=7.1 Hz, 6H), 1.02 (t, J=7.3 Hz, 3H).

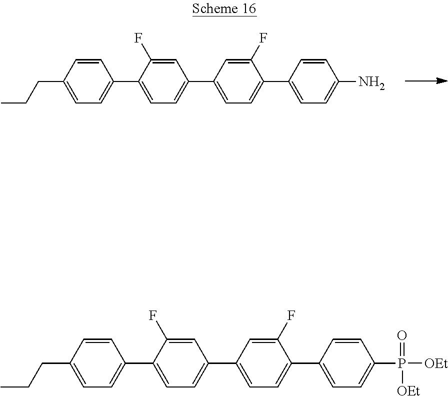 [SMILES: CCCc1ccc(-c2ccc(-c3ccc(-c4ccc(N)cc4)c(F)c3)cc2F)cc1 ; CCCc1ccc(-c2ccc(-c3ccc(-c4ccc(P(=O)(OCC)OCC)cc4)c(F)c3)cc2F)cc1]

### Synthesis of LCC of Structure (M12) (SML-402)

Scheme 17 shows a general outline of the second step in the synthesis of LCC of structure (M12), specifically, (4′-Propyl[1,1′-biphenyl]-4-yl)-boronic acid (24.0 g, 0.10 mol), 4-bromo-2,6-difluoroaniline (31.2 g, 0.15 mol), and sodium carbonate (31.8 g, 0.30 mol) were suspended in a mixture of isopropanol (200 mL), water (220 mL), and toluene (280 mL). Bis[tricyclohexylphosphino]palladium (II) chloride (2.0 g, 2.8 mmol) and hydrazine hydrate (0.1 mL, 80%) were added and the mixture was stirred for 8 h at 80° C. After cooling down to −15° C., the precipitate was filtered off and washed with cold water. Further purification by column chromatography (silica gel, toluene/petroleum ether: 2/8) and recrystallization from toluene gave 2,6-difluoro-4-[4-(4-propylphenyl)-phenyl] aniline (20.7 g, 64.0 mmol, HPLC: 98.0%) was obtained as a colorless solid in 64% yield. ¹H-NMR (CDCl₃, 400 MHz): δ=7.65-7.60 (m, 2H), 7.57-7.52 (m, 4H), 7.29-7.23 (m, 2H), 7.15-7.11 (m, 2H), 3.78 (s, 2H), 2.64 (t, J=7.5 Hz, 2H), 1.74-1.62 (m, 2H), 0.98 (t, J=7.3 Hz, 3H). EI-MS: m/z: 323.1. DSC: 185° C. I. TGA: T_{5% loss}=247° C.

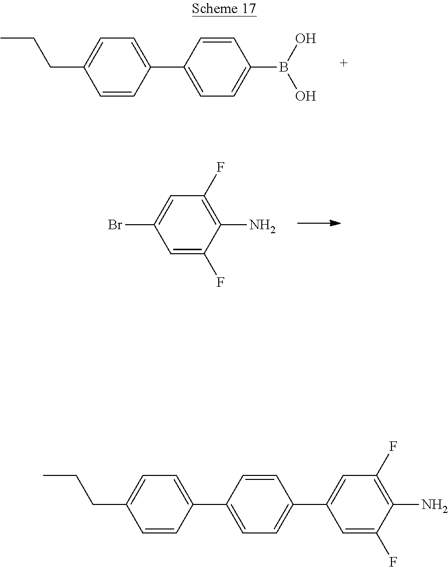 [SMILES: CCCc1ccc(-c2ccc(B(O)O)cc2)cc1 ; Nc1c(F)cc(Br)cc1F ; CCCc1ccc(-c2ccc(-c3cc(F)c(N)c(F)c3)cc2)cc1]

### Synthesis of LCC of Structure (M13) (SML-403)

Scheme 18 shows a general outline of the second step in the synthesis of LCC of structure (M13), specifically, 3,5-Difluoro-4′-propyl[1,1′-biphenyl]-4-yl-boronic acid (27.6 g, 0.10 mol), 4-bromo-aniline (25.8 g, 0.15 mol), and sodium carbonate (31.8 g, 0.30 mol) were suspended in a mixture of isopropanol (200 mL), water (220 mL), and toluene (280 mL). Bis[tricyclohexylphosphino]palladium (II) chloride (2.0 g, 2.8 mmol) and hydrazine hydrate (0.1 mL, 80%) were added and the mixture was stirred for 6 h at 80° C. After cooling down to room temperature, the phases were separated, and the aqueous phase was extracted with toluene. Further purification by column chromatography (silica gel, toluene) and recrystallization from toluene gave 2,6-difluoro-4-[4-(4-propylphenyl)-phenyl] aniline (10.3 g, 31.8 mmol, HPLC: 99.1%) was obtained as a colorless solid in 31% yield. ¹H-NMR (DMSO-d₆, 300 MHz): δ=7.68 (d, J=8.2 Hz, 2H), 7.49-7.41 (m, 2H), 7.30 (d, J=8.3 Hz, 2H), 7.16-7.12 (m, 2H), 6.70-6.62 (m, 2H), 5.34 (s, 2H), 2.60 (t, J=7.5 Hz, 2H), 1.72-1.49 (m, 2H), 0.92 (t, J=7.3 Hz, 3H). EI-MS: m/z: 323.1. DSC: 105° C. (99° C.) I. TGA: T_{5% loss}=239° C.

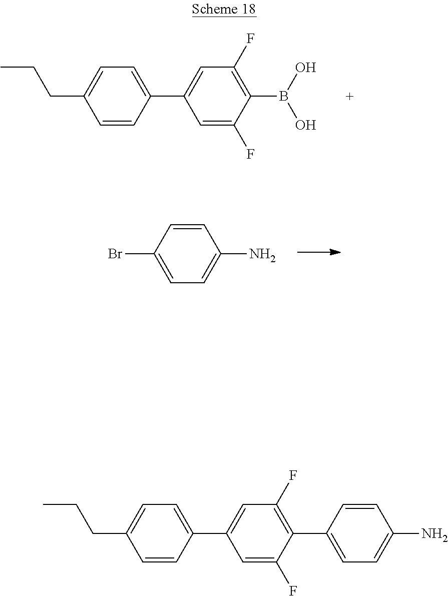 [SMILES: CCCc1ccc(-c2cc(F)c(B(O)O)c(F)c2)cc1 ; Nc1ccc(Br)cc1 ; CCCc1ccc(-c2cc(F)c(-c3ccc(N)cc3)c(F)c2)cc1]

Synthesis of LCC of structure (M14) (SML-404)

### Step 3 in Synthesis of LCC of Structure (M14)

Scheme 19 shows a general outline of the third step in the synthesis of LCC of structure (M14), specifically, [2-Fluoro-4-[3-fluoro-4-(4-propylphenyl)phenyl] phenyl] boronic acid (18.5 g, 52.5 mmol), 4-bromoaniline (9.5 g, 55.0 mmol), and sodium carbonate (17.4 g, 0.16 mol) were suspended in a mixture of isopropanol (200 mL), water (220 mL), and toluene (280 mL). Bis[tricyclohexylphosiphino]palladium (II) chloride (2.5 g, 3.4 mmol) and hydrazine hydrate (0.1 mL, 80%) were added and the mixture was stirred for 6 h at 80° C. After cooling down to 0° C., the precipitate was filtered off and washed with cold toluene. Additionally, the filtrate was extracted with toluene. The combined fractions were recrystallized from toluene/isopropanol and further purified by column chromatography (silica gel, toluene). 2′-fluoro-4′-{2-fluoro-4′-propyl-[1,1′-biphenyl]-4-yl}-[1,1′-biphenyl]-4-amine (16.0 g, 40.1 mmol, HPLC: 98.9%) was obtained as a colorless solid in 76% yield. ¹H-NMR (CDCl₃, 400 MHz): δ=7.55-7.36 (m, 10H), 7.30-7.26 (m, 2H), 6.81-6.75 (m, 2H), 3.79 (s, 2H), 2.65 (dd, J=8.5 Hz, J=6.8 Hz, 2H), 1.77-1.62 (m, 2H), 0.99 (t, J=7.3 Hz, 3H).

EI-MS: m/z: 399.2. DSC: 139° C. N, 315.2° C. I. TGA: T_{5% loss}=335° C.

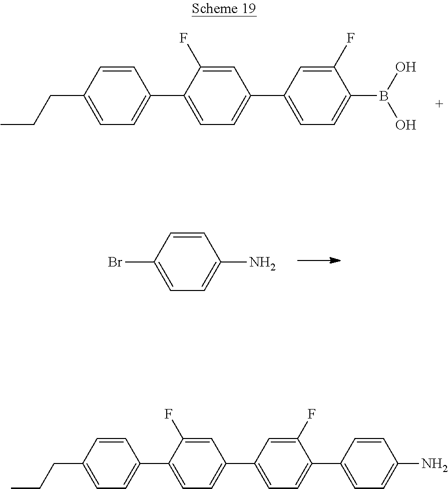 [SMILES: CCCc1ccc(-c2ccc(-c3ccc(B(O)O)c(F)c3)cc2F)cc1 ; Nc1ccc(Br)cc1 ; CCCc1ccc(-c2ccc(-c3ccc(-c4ccc(N)cc4)c(F)c3)cc2F)cc1]

### Step 2 in Synthesis of LCC of Structure (M14)

Scheme 20 shows a general outline of the second step in the synthesis of LCC of structure (M14), specifically, n-Butyllithium (30 mL, 80.0 mmol, 2.7 M in heptane) was added dropwise to a solution of 1-fromo-2-fluoro-4-[3-fluoro-4-(4-propylphenyl)phenyl] benzene (25.0 g, 64.6 mmol) in THF (970 mL) at −95° C. The reaction mixture was stirred for 2 h at −95° C. before it allowed to warm up to −80° C. The resulting solution was again cooled down to −95° C. and trimethyl borate (9.3 g, 90.0 mmol) was added. After stirring for 30 min at −95° C., the solution allowed to slowly warm up to room temperature. Water (200 mL) was added, and the aqueous phase was adjusted to pH 2 by adding hydrochloric acid. After stirring for 1 h at room temperature, the aqueous phase was extracted with diethyl ether and the combined organic phases were washed with diluted hydrochloric acid. The crude product was used without further purification in the next step.

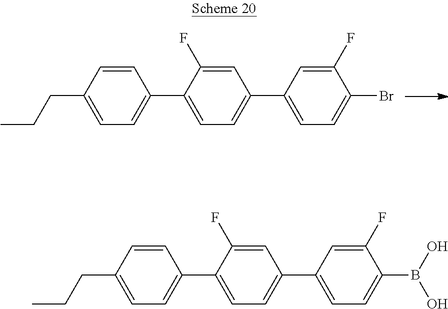 [SMILES: CCCc1ccc(-c2ccc(-c3ccc(Br)c(F)c3)cc2F)cc1 ; CCCc1ccc(-c2ccc(-c3ccc(B(O)O)c(F)c3)cc2F)cc1]

### Step 1 in Synthesis of LCC of Structure (M14)

Scheme 21 shows a general outline of the first step in the synthesis of LCC of structure (M14), specifically, 4′-Propyl-2-fluoro-4-biphenylboronic acid (30.0 g, 0.12 mol), 4-bromo-3-fluoro-iodobenzene (54.2 g, 0.18 mol), and sodium carbonate (31.8 g, 0.30 mol) were suspended in a mixture of isopropanol (200 mL), water (220 mL), and toluene (280 mL). Bis[tricyclohexylphosiphino]palladium (II) chloride (2.0 g, 2.8 mmol) and hydrazine hydrate (0.1 mL, 80%) were added and the mixture was stirred for 3 d at room temperature. After the addition of Bis[tricyclohexylphosiphino]palladium (II) chloride (1.5 g, 2.1 mmol), the reaction mixture was stirred for 8 h at 80° C. After cooling down to room temperature, the organic phase was separated, and the aqueous phase was extracted with toluene. The combined organic phases were washed with saturated NaCl solution and dried over sodium sulphate. Further purification by column chromatography (silica gel, petroleum ether) and recrystallization from petroleum ether gave 1-fromo-2-fluoro-4-[3-fluoro-4-(4-propylphenyl)phenyl] benzene as a colorless solid (41.3 g, 106.6 mmol) in 92% yield.

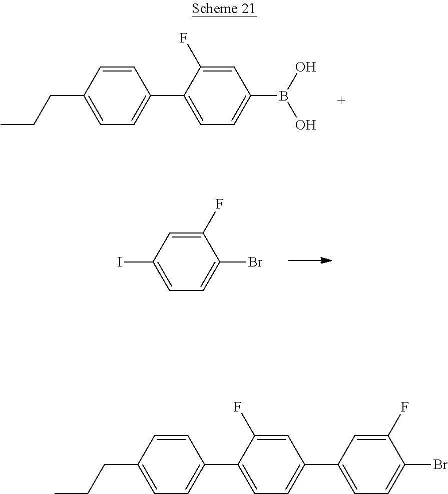 [SMILES: CCCc1ccc(-c2ccc(B(O)O)cc2F)cc1 ; Fc1cc(I)ccc1Br ; CCCc1ccc(-c2ccc(-c3ccc(Br)c(F)c3)cc2F)cc1]

### Synthesis of LCC of Structure (M15) (SML-405)

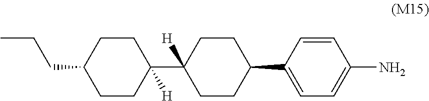 [SMILES: CCC[C@H]1CC[C@H]([C@H]2CC[C@H](c3ccc(N)cc3)CC2)CC1]

The LLC of structure (M15),4-[(Trans, trans)-4′-propyl[1,1′-bicyclohexyl]-4-yl]-benzamine was made according to the procedure of Kanie, Kiyoshi et al., Chemistry Letters 1995, 24, 683.

### Synthesis of LCC of Structure (M16) (SML-406)

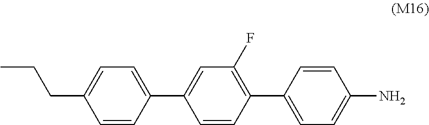 [SMILES: CCCc1ccc(-c2ccc(-c3ccc(N)cc3)c(F)c2)cc1]

The LLC of structure (M16), 2′-Fluoro-4″-propyl-[1,1′:4′,1″-terphenyl]-4-amine was made according to the procedure of Wang, Chun-Chih et al., U.S. Pat. No. 8,741,176 B2.

### Synthesis of LCC of Structure (M17) (SML-407)

The LLC of structure (M17), 2′-Fluoro-4″-pentyl-[1,1′:4′,1″-terphenyl]-4-amine was synthesized according to the procedure for 2′-fluoro-4″-propyl-[1,1′:4′,1″-Terphenyl]-4-amine which is described in Wang, Chun-Chih et al., U.S. Pat. No. 8,741,176 B2.

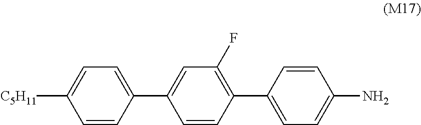

What is claimed is:

1. A patterned liquid crystal monolayer structure (100) on a patterned substrate (900), comprising:
the patterned substrate (900) comprising a first surface area (500) having a liquid crystal compound (300) adsorbed thereon as a self-assembled monolayer (SAM) (200) and a second surface area (400) free of the liquid crystal compound (300);
wherein said liquid crystal compound (300) consists of a cylindrical linear organic liquid crystal core structure comprising at least one 1,4-phenylene moiety (700) to which is attached at one end a linear alkyl group comprising at least two carbons (600) and at the other end a polar anchor group (800) selected from the group consisting of a phosphonate ester comprising moiety, a phosphonic acid comprising moiety, a thiol comprising moiety, an amino comprising moiety, at least one alkylenehydroxy comprising moiety, and an alkyl-polyol comprising moiety;
wherein in said self-assembled monolayer (200), said liquid crystal compound (300) is arranged in the self-assembled monolayer (200) so that each liquid crystal compounds in said self-assembled monolayer (200) are parallel to each other and pointing in the same direction, perpendicular to said patterned substrate (900) and attached to surface area (500) only through said polar anchor group (800); and
wherein either,
when said polar anchor group (800) is an alkyl-polyol coordination moiety or at least one alkylenehydroxy comprising moiety, said surface area (500) to which said self-assembled monolayer (200) is attached, is a non-metallic inorganic silicon compound-based surface area and said surface area (400) free of the liquid crystal compound (300) is a metallic surface area or,
when said polar anchor group is selected from the group consisting of a phosphonate ester comprising moiety, a phosphonic acid comprising moiety, a thiol comprising moiety, and an amino comprising moiety, said surface area (500) to which said self-assembled monolayer (200) is attached, is a metallic surface area and said surface area (400) free of the liquid crystal compound (300) is a non-metallic inorganic silicon compound-based surface area.

2. The patterned monolayer structure on a substrate of claim 1, wherein said linear alkyl group (600) is a C-2 to C-5 alkyl.

3. The patterned monolayer structure on a substrate of claim 1, wherein said cylindrical linear organic liquid crystal core structure comprising at least two 1,4-phenylene moieties (700) is selected from the group consisting of structures (I), (II), (IIa), (III), (IV), (V), (VI), (VII), and (VIII), wherein ** is the attachment point of said linear alkyl group (600) and * is the attachment point of said polar anchor group (800), R₁, and R₂ are individually selected from H, a C-1 to C-2 alkyl and F,

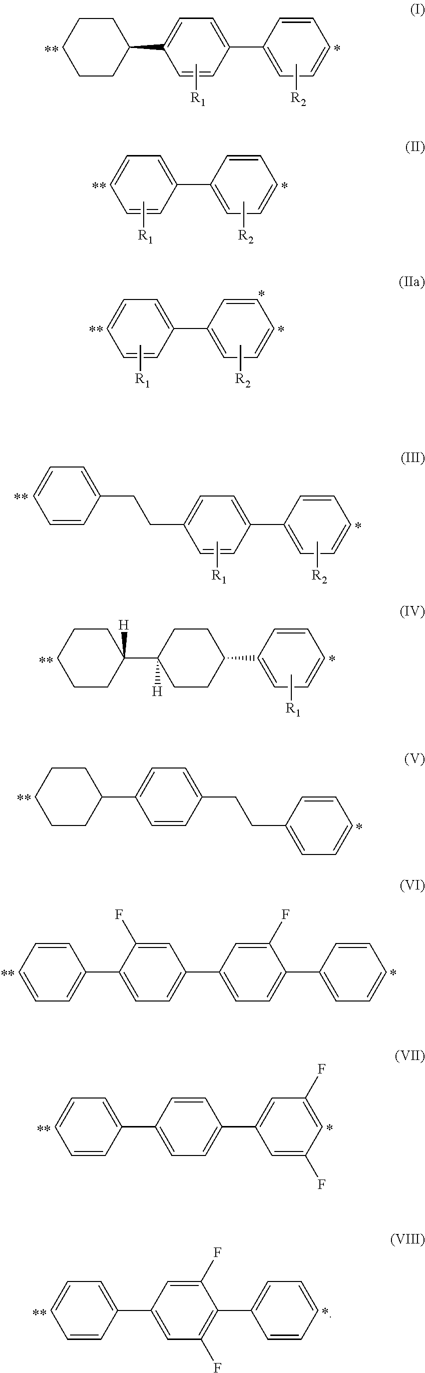

4. The patterned monolayer structure on a substrate of claim 1, wherein said polar anchor group (800) is selected from the group consisting of structure (Ip), structure (Ipa), structure (IIp), and structure (IIpa), wherein L₁, and L₂ are individually selected from a C-2 to C-4 linear alkylene spacer, and *** is the attachment point of said polar anchor group to said liquid crystal compound,

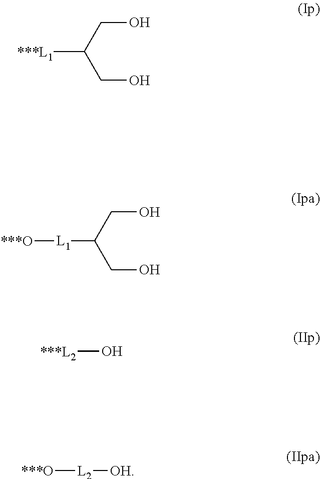

5. The patterned monolayer structure on a substrate of claim 1, wherein said polar anchor group (800) is selected from the group consisting of structure (IIIp), structure (IVp), structure (Vp), structure (VIp), structure (VIIp), structure (VIIIp) and structure (IXp) wherein L₃, L₄, L₅, L₆, and L₇ are individually selected from a C-2 to C-4 linear alkylene spacer, *** is the attachment point of said polar anchor group (800) to said liquid crystal compound (300), Rₚ₁ is a C-1 to C-4 alkoxy, and Rₚ₂ is a C-1 to C-4 alkyl or a C-1 to C-4 alkoxy,

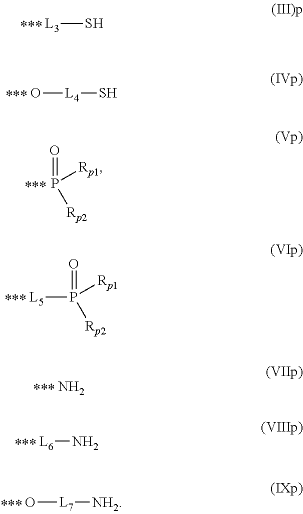

6. The patterned monolayer structure on a substrate of claim 1, wherein said liquid crystal compound (300) is one selected from group consisting of one having structures (M1), (M2), (M3), (M4), (M5), and (M7),

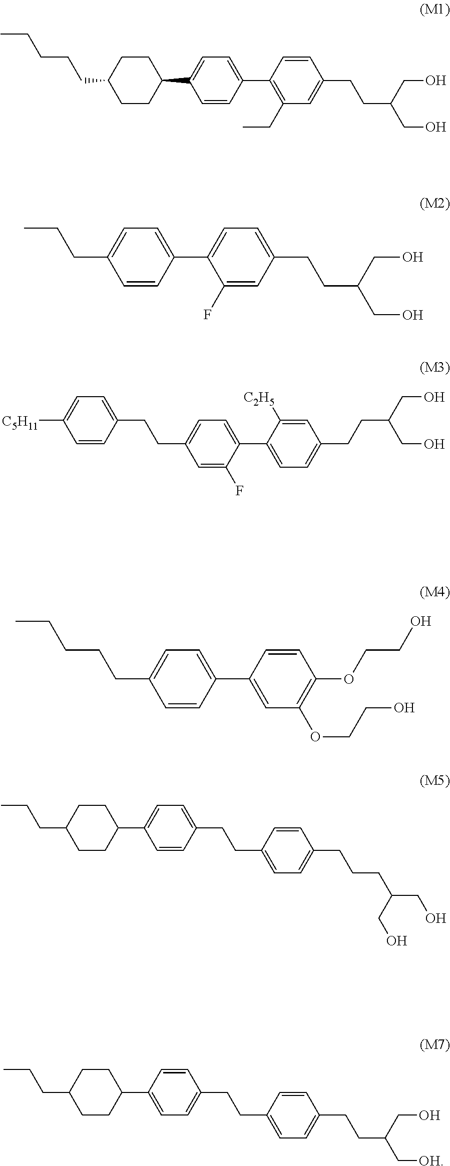

7. The patterned monolayer structure on a substrate of claim 1, wherein said liquid crystal compound (300) is one selected from group consisting of one having structures (M8), (M9), (M10), (M11), (M12), (M13) and (M14),

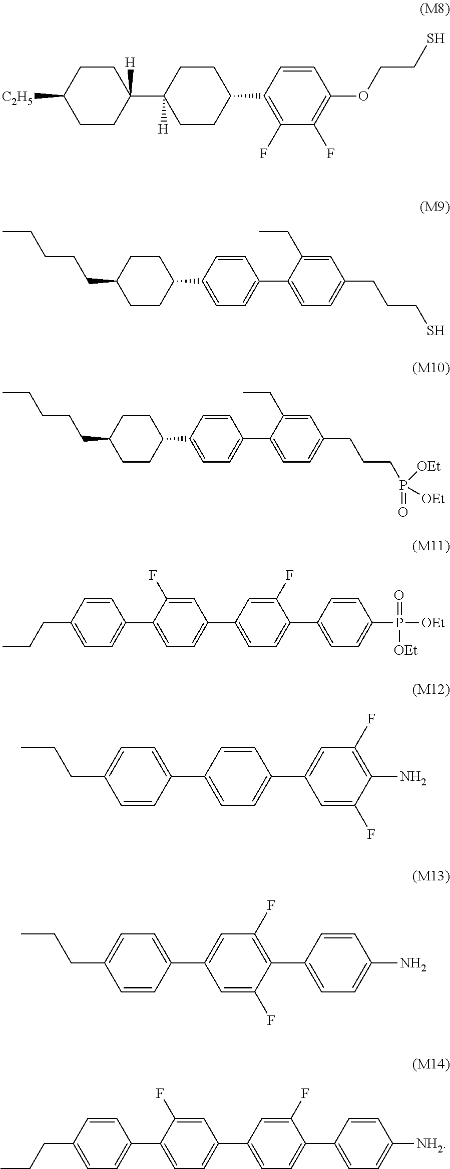

8. The patterned monolayer structure on a substrate of claim 1 wherein said surface area (500) overlaid with a SAM is a non-metallic inorganic silicon compound-based surface area selected from the group consisting of silicon oxide (SiO₂), silicon with a native oxide, silicon nitride (SiN) and silicon oxynitride (SiON) and said surface area (400) which is bare, is a metal which is selected from the group consisting of tungsten, gold, silver, copper, cobalt, ruthenium, zirconium, titanium, and hafnium.

9. The patterned monolayer structure on a substrate of claim 1 wherein said surface area (500) overlaid with a SAM is a metal selected from the group consisting of tungsten, gold, silver, copper, cobalt, ruthenium, zirconium, titanium, and hafnium and said surface area (400) which is bare, is a non-metallic inorganic silicon compound-based surface area which is selected from the group the group consisting of silicon oxide (SiO₂), silicon with a native oxide, silicon nitride (SiN) and silicon oxynitride (SiON).

10. A composition comprising a liquid crystal compound (300) and an organic spin casting solvent, wherein said liquid crystal compound consists of a cylindrical linear organic liquid crystal core structure comprising at least one 1,4-phenylene moieties (700) to which is attached at one end a linear alkyl group with at least two carbons (600) and at the other end a polar anchor group (800).

11. The composition of claim 10, wherein said linear alkyl group (600) is a C-2 to C-5 alkyl.

12. The composition of claim 10, wherein said cylindrical linear organic liquid crystal core structure comprising at least two 1,4-phenylene moieties (700) is selected from the group consisting of structures (I), (II), (IIa), (III), (IV), (V), (VI), (VII), and (VIII), wherein ** is the attachment point of said linear alkyl group (600) and * is the attachment point of said polar anchor group (800), R₁, and R₂ are individually selected from H, a C-1 to C-2 alkyl and F,

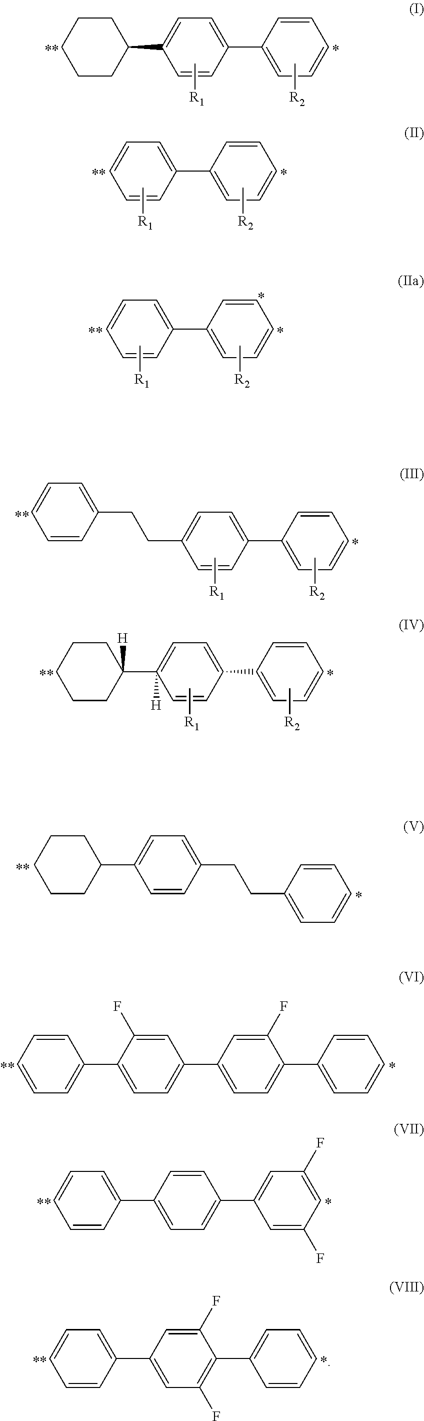

13. The composition of claim 10, wherein said polar anchor group (800) is selected from the group consisting of structure (Ip), structure (Ipa), structure (IIp), and structure (IIpa), wherein L₁ and L₂ are individually selected from a C-2 to C-4 linear alkylene spacer, *** is the attachment point of said polar anchor group (800) to said liquid crystal compound (300),

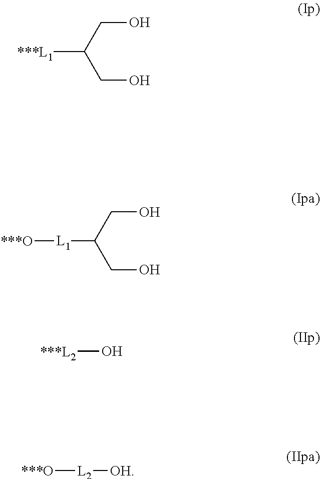

14. The composition of claim 10, wherein said polar anchor group (800) is selected from the group consisting of structure (IIIp), structure (IVp), structure (Vp), structure (VIp), structure (VIIp), structure (VIIIp) and structure (IXp) wherein L₃, L₄, L₅, L₆, and L₇ are individually selected from a C-2 to C-4 linear alkylene spacer, *** is the attachment point of said polar anchor group (800) to said liquid crystal compound (300), Rₚ₁ is a C-1 to C-4 alkoxy, and Rₚ₂ is a C-1 to C-4 alkyl or a C-1 to C-4 alkoxy,

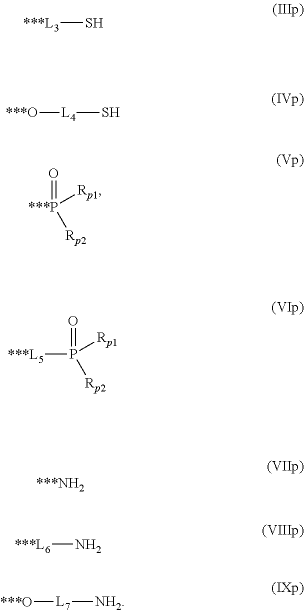

15. The composition of claim 10, wherein said liquid crystal compound (300) is selected from the group consisting of one having structures (M1), (M2), (M3), (M4), (M5), and (M7),

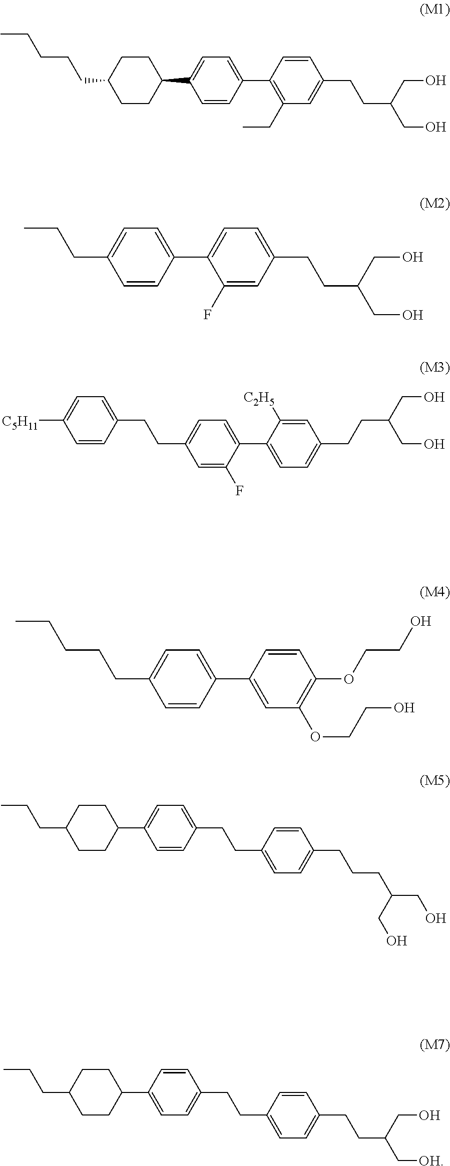

16. The composition of claim 10, wherein said liquid crystal compound (300) is selected from the group consisting of one having structures (M8), (M9), (M10), (M11), (M12), (M13) and (M14),

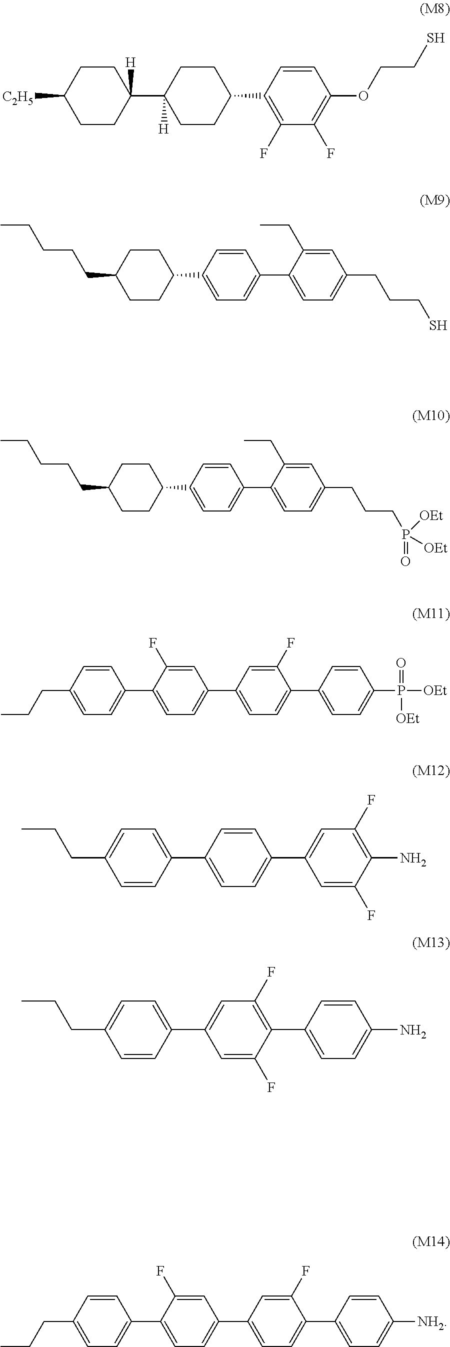

17. The composition of claim 10, wherein said liquid crystal compound (300) is one having structure (M4),

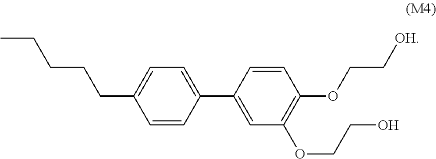 [SMILES: CCCCCc1ccc(-c2ccc(OCCO)c(OCCO)c2)cc1]

18. The composition of claim 10, wherein said liquid crystal compound (300) is one selected from the group consisting of one having structures (M8), (M9), (M12), (M13) and (M14),

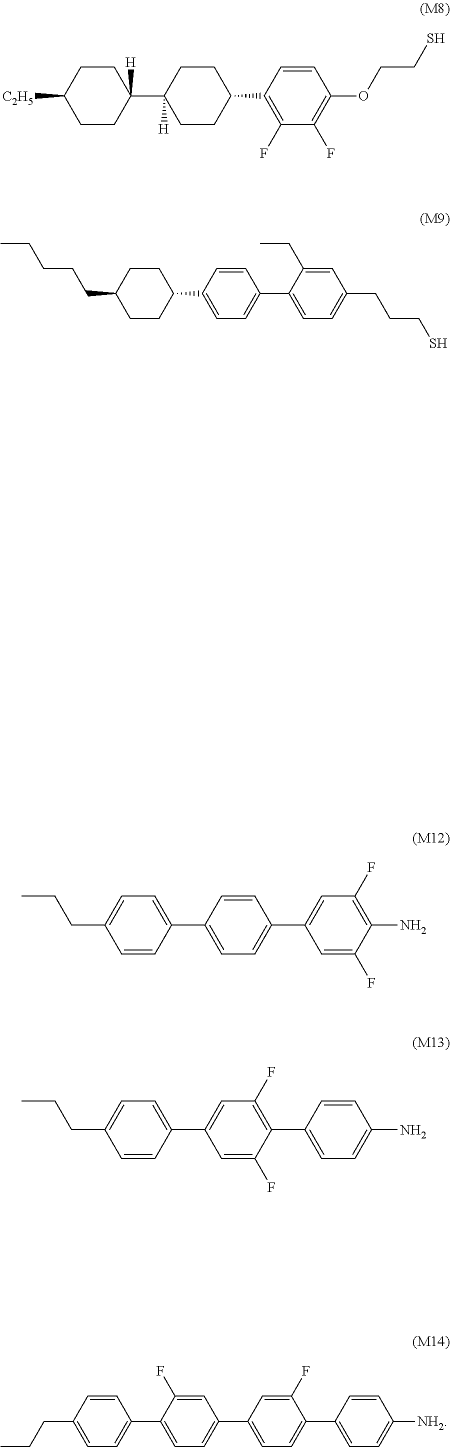 [SMILES: CC[C@H]1CC[C@H]([C@H]2CC[C@H](c3ccc(OCCS)c(F)c3F)CC2)CC1 ; CCCCC[C@H]1CC[C@H](c2ccc(-c3ccc(CCCS)cc3CC)cc2)CC1 ; CCCc1ccc(-c2ccc(-c3cc(F)c(N)c(F)c3)cc2)cc1 ; CCCc1ccc(-c2cc(F)c(-c3ccc(N)cc3)c(F)c2)cc1 ; CCCc1ccc(-c2ccc(-c3ccc(-c4ccc(N)cc4)c(F)c3)cc2F)cc1]

19. The composition of claim 10, wherein said liquid crystal compound (300) is one selected from the group consisting of one having structures (M10), and (M11),

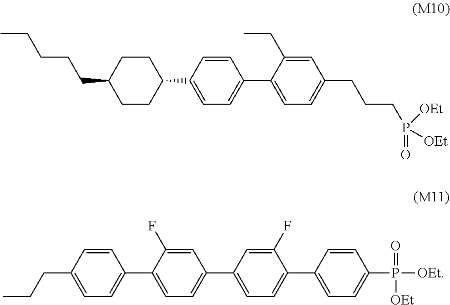 [SMILES: CCCCC[C@H]1CC[C@H](c2ccc(-c3ccc(CCCP(=O)(OCC)OCC)cc3CC)cc2)CC1 ; CCCc1ccc(-c2ccc(-c3ccc(-c4ccc(P(=O)(OCC)OCC)cc4)c(F)c3)cc2F)cc1]

20. The composition of claim 10, wherein said organic spin coating solvent is a single organic solvent or a mixture of at least two organic solvents selected from the group consisting of a glycol ether derivative selected from ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA),
a carboxylate of a mono-basic acid selected from ethyl acetate, n-butyl acetate and amyl acetate,
a carboxylate of di-basic acids selected from diethyloxylate and diethylmalonate,
a dicarboxylates of a glycol selected from ethylene glycol diacetate and propylene glycol diacetate,
a hydroxy carboxylate selected from methyl lactate, ethyl lactate (EL), ethyl glycolate, and ethyl-3-hydroxy propionate,
a ketone ester selected from methyl pyruvate and ethyl pyruvate,
an alkyloxycarboxylic acid ester selected from methyl 3-methoxypropionate, ethyl 3-ethoxypropionate, ethyl 2-hydroxy-2-methylpropionate, and methylethoxypropionate,
a ketone derivative selected from methyl ethyl ketone, acetyl acetone, cyclopentanone, cyclohexanone and 2-heptanone, diacetone alcohol methyl ether,
a ketone alcohol derivative selected from acetol and diacetone alcohol,
a ketal or acetal selected from 1,3 dioxalane and diethoxypropane, butyrolactone,
an amide selected from dimethylacetamide and dimethylformamide, and
anisole.

21. The composition of claim 10, wherein said liquid crystal compound is present at a loading of about 0.5 wt. % to about 2.0 wt. %.

22. The composition of claim 10, wherein said organic spin coating solvent is a mixture of PGME and PGMEA.

23. The composition of claim 10, wherein said organic spin coating solvent is a mixture of 70 wt. % PGME and 30 wt. % PGMEA.

24. The composition of claim 10, wherein said organic spin coating solvent is a single organic solvent or a mixture of at least two organic solvents selected from the group consisting of
a glycol ether derivative selected from ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA),
a carboxylate of a mono-basic acid selected from ethyl acetate, n-butyl acetate and amyl acetate,
a carboxylate of di-basic acids selected from diethyloxylate and diethylmalonate,
a dicarboxylates of a glycol selected from ethylene glycol diacetate and propylene glycol diacetate,
a hydroxy carboxylate selected from methyl lactate, ethyl lactate (EL), ethyl glycolate, and ethyl-3-hydroxy propionate,
a ketone ester selected from methyl pyruvate and ethyl pyruvate,
an alkyloxycarboxylic acid ester selected from methyl 3-methoxypropionate, ethyl 3-ethoxypropionate, ethyl 2-hydroxy-2-methylpropionate, and methylethoxypropionate,
a ketone derivative selected from methyl ethyl ketone, acetyl acetone, cyclopentanone, cyclohexanone and 2-heptanone,
diacetone alcohol methyl ether,
a ketone alcohol derivative selected from acetol and diacetone alcohol,
a ketal or acetal selected from 1,3 dioxalane and diethoxypropane,
butyrolactone,
an amide selected from dimethylacetamide and dimethylformamide, and
anisole.

25. The composition of claim 10, wherein said liquid crystal compound is present at a loading of about 0.5 wt. % to about 2.0 wt. %.

26. The composition of claim 10, wherein said organic spin coating solvent is a mixture of PGME and PGMEA.

27. The composition of claim 10, wherein said organic spin coating solvent is a mixture of 70 wt. % PGME and 30 wt. % PGMEA.

28. A process for forming a self-assembled monolayer (SAM) of a liquid crystal (LC) selectively on nonmetallic areas in a mixed substrate which comprises both metallic and nonmetallic areas, the process comprising the steps:
i) spin coating on a mixed substrate the composition of claim 10, where said polar anchor group (800) is an alkyl-polyol coordination moiety or at least one alkylenehydroxy comprising moiety, and where said nonmetallic areas are selected from silicon dioxide, silicon with native oxide, silicon nitride, and silicon oxynitride, and said metallic areas are selected from the group consisting of tungsten, gold, silver, copper, cobalt, ruthenium, zirconium, titanium, and hafnium,
ii) baking at a temperature ranging from about 150° C. to about 180° C. for about 2 min to about 10 min under an inert gas,
iii) rinsing with an organic spin coating solvent,
iv) air drying substrate,
v) repeat steps i) to iv) two times, to obtain self-assembled monolayer of liquid crystal on both the metallic and non-metallic areas,
vi) rinsing the substrate with a dilute aqueous solution of an acid to selectively remove the self-assembled monolayer of liquid crystal on the metallic areas,
vii) rinsing the substrate with water and air drying to obtain a substrate in where the non-metallic areas only have a SAM of a LC.

29. A process for selective atomic layer deposition on metallic areas of a mixed substrate comprising both metallic and non-metallic areas, the process comprising the steps:
ia) producing a mixed substrate in which non-metallic areas have a self-assembled monolayer of liquid crystal according to claim 28,
iia) using an atomic layer deposition technique, deposit a metal oxide more selectively on the metallic areas which do not have a self-assembled monolayer of liquid crystal.

30. The process of claim 29, wherein said atomic layer deposition technique is one employing deposition of about 10 to about 50 cycles of deposition, wherein each cycle employs a vapor treatment with (MeCp)₂Hf(OMe)Me for about 1 to about 5 s followed by a treatment with N₂ for about 5 s to about 15 s, a treatment with H₂O for about 1 s to about 5 s, and a treatment with N₂ for about 5 s to about 15 s at a temperature of about 250° C. to about 350° C.

31. A process for forming a self-assembled monolayer (SAM) of a liquid crystal (LC) selectively on metallic areas in a mixed substrate which comprises both metallic and nonmetallic areas, the process comprising the steps:
ib) spin coating on a mixed substrate the composition of claim 14, 16, 18, 19, 23, 24, 25, and 26, where a polar anchor group (800) is selected from the group consisting of a phosphonate ester comprising moiety, a phosphonic acid comprising moiety, a thiol comprising moiety, and an amino comprising moiety, and wherein said nonmetallic areas are selected from silicon dioxide, silicon with native oxide, silicon nitride, and silicon oxynitride, and said metallic areas are selected from the group consisting of tungsten, gold, silver, copper, cobalt, ruthenium, zirconium, titanium, and hafnium,
iib) baking at a temperature ranging from about 150° C. to about 180° C. for about 2 min to about 10 min under an inert gas,
iiib) rinsing with an organic spin coating solvent,
ivb) air drying substrate,
vb) repeat steps ib) to ivb) two times,
vib) air drying substrate, to obtain a self-assembled monolayer of liquid crystal only on the metallic areas of said substrate.

32. A process for selective atomic layer deposition on non-metallic areas of a mixed substrate comprising both metallic and non-metallic areas, the process comprising the steps:
ic) producing a mixed substrate in which metallic areas have a self-assembled monolayer of liquid crystal according to claim 31,
iic) using an atomic layer deposition technique, deposit a metal oxide more selectively on the non-metallic areas which do not have a self-assembled monolayer of liquid crystal.

33. The process of claim 32, where said atomic layer technique is one employing deposition of about 10 to about 50 cycles of deposition, wherein each cycle employs a vapor treatment with (MeCp)₂Hf(OMe)Me for about 1 to about 5 s followed by a treatment with N₂ for about 5 s to about 15 s, a treatment with H₂O for about 1 s to about 5 s, and a treatment with N₂ for about 5 s to about 15 s at a temperature of about 250° C. to about 350° C.

34. (canceled)
